(19) United States
(12) Patent Application Publication
(10) Pub. No.: US 2016/0130606 A1
(43) Pub. Date: May 12, 2016

(54) FREEZE-DRIED POLYELECTROLYTE COMPLEXES THAT MAINTAIN SIZE AND BIOLOGICAL ACTIVITY

(71) Applicant: POLYVALOR, SOCIÉTÉ EN COMMANDITE, Montréal (CA)

(72) Inventors: Michael D. BUSCHMANN, Montréal (CA); Marc LAVERTU, Pointe-Claire (CA); Daniel VEILLEUX, Montréal (CA)

(21) Appl. No.: 14/897,424

(22) PCT Filed: Jun. 9, 2014
(86) PCT No.: PCT/CA2014/000490

§ 371 Date: Dec. 10, 2015

Related U.S. Application Data
(60) Provisional application No. 61/833,010, filed on Jun. 10, 2013.

(51) Int. Cl. C12N 15/87; A61K 48/00

(52) U.S. Cl. CPC C12N 15/87; A61K 48/0041

(57) ABSTRACT

The present invention relates to a polyelectrolyte complex composition comprising a polymer, a nucleic acid molecule, a lyoprotectant, and a buffer. The polyelectrolyte complex composition preserving the biological activities of the polyelectrolyte complex following freeze-drying and rehydration.

### CROSS-REFERENCE TO RELATED APPLICATIONS

This application claims the benefit of and priority to U.S. provisional patent application No. 61/833,010, filed on Jun. 10, 2013, the content of which is herein incorporated in its entirety by reference.

### TECHNICAL FIELD

The present invention relates to polyelectrolyte complex compositions that have increased stability in solution and that have improved resistance to physical or chemical degradation over long-term storage; methods for obtaining such polyelectrolyte complex compositions as well as the use of these polyelectrolyte complex compositions for delivery of nucleic acids.

### BACKGROUND INFORMATION

Significant efforts have been made in recent years to develop nanoparticle compositions for delivery of therapeutic drugs, such as but not limited to proteins, peptides, deoxyribonucleic acids (DNA), such as plasmid (pDNA) and oligodeoxynucleotides (ODN), and ribonucleic acids, such as small interfering ribonucleic acids (siRNA) and small hairpin ribonucleic acids (shRNA). However, these colloidal compositions were shown to have limited stability in solution and to be prone to physical or chemical degradation over long-term storage [1].

Dehydration of these compositions by freeze-drying, also known as lyophilization, is used to increase their long-term stability [2-4]. This process consists of 3 main steps: freezing, primary drying, and secondary drying. It offers the possibility of rehydrating dried compositions in reduced volumes to increase the active agent concentration and reach therapeutic dosages. This is of particular interest in the case of self-assembling polyelectrolyte complexes compositions formed between a polycation and a nucleic acid (NA) which require preparation in dilute conditions to produce small uniformly sized nanoparticles [1].

Addition of lyoprotectants to compositions is generally required to prevent irreversible aggregation and loss of functionality of nanoparticles in solution upon lyophilization [4, 5]. Freeze-thaw studies allow to identify potential lyoprotectants to be used for a given composition [6, 7]. Disaccharides (such as sucrose, trehalose, lactose, etc.), oligosaccharides/polysaccharides (such as cellulose, dextran, etc.), polymers (such as PEG, PVP, etc.), etc. have been used as lyoprotectants to stabilize compositions for long-term storage [3, 4]. Trehalose would also be an excellent nanoparticle lyoprotectant [4, 11-13]. Yet, freeze-dried amorphous disaccharides were found to crystallize more readily than polysaccharides upon storage at high temperatures, increasing risks of complex aggregation [14], though polysaccharides were found to be less efficient lyoprotectants due to their bulkiness [15, 16]. Oligosaccharides could be superior stabilizers given they possess the advantageous properties of both disaccharides and polysaccharides for optimal stabilization of complexes [17]. Low molecular weight dextrans have proven efficient at preserving polyplex physico-chemical properties and functionality upon freeze-drying and rehydration, while preserving higher cell viability than sucrose during in vitro and in vivo studies [15].

Buffers may be used to stabilize the pH and prevent nanoparticle acid hydrolysis during the cryoconcentration of solutes occurring through the freezing phase of the lyophilization process [2]. Buffers used during freeze-drying must be chosen with care as some crystallize or precipitate during freezing (phosphate, succinate or tartrate salts), causing pH shifts reaching up to 4 units [2, 20-23]. Tg′, which refers to the glass transition temperature of maximally cryocentrated solutions, is an important parameter to consider when selecting excipients for freeze-drying; it is a good estimate of the highest temperature at which primary drying can be performed without affecting the final product. Sodium citrate is a non crystallizing buffer adequate for use in freeze-dried injectable compositions given its higher Tg′ at various pH values [22] and its pKₐ close to neutrality (pKₐ₃=6.4) [24]. L-histidine could also be adequate given one of its three pKₐ values is at 6.1, it exhibits little crystallization upon freeze-drying at pH of 5.5 to 6.5, and it has a high Tg′ (−33° C.) [12, 25]. Use of excipients, mostly lyoprotectants and some buffers, has been characterized for the development of freeze-dried polyelectrolyte complex compositions formed with Poly(D,L lactic-co-glycolic acid) (PLGA) (U.S. 2011/262490) [26-28], poly(l-lysine) (PLL) [29], polylactic acid (PLA) (U.S. 2011/0275704) [30], gelatin [31], polyethyleneimine (PEI) [7, 15, 17, 32-37].

PEI-based nanoparticle systems are the most characterized in freeze-drying. Several disaccharides proved efficient at lyoprotecting PEI/NA nanoparticles during freeze-drying. A prior lyoprotectant screening study showed that relatively high concentrations of sucrose (equivalent to 37.5% (w/v) for compositions containing 50 μg of DNA per mL) were required to preserve 70 kDa (weight average molecular weight (M_{w})) branched PEI/DNA particle size upon freeze-thawing, although sharp decreases in zeta potential and transfection efficiencies resulted [32]. More recent work showed that 25 kDa (M_{w}) branched PEI/DNA complexes could be freeze-dried in much lower concentrations of sucrose, lactose or trehalose (equivalent to 1.25% (w/v) for compositions containing 50 μg of DNA per mL) without particle aggregation or loss of transfection in vitro, acceptable increases in zeta potentials (10 to 20 mV), and highest transfection efficiency in vivo with lactose compositions [33]. Mannitol, as well as sucrose or trehalose, could also be used to prevent aggregation and loss of efficiency of PEI/DNA complexes upon lyophilization, but no lyoprotectant was required for PEI/ODN or ribozymes complexes [34].

Previous studies established that high sucrose/DNA weight ratios were required to stabilize nanoparticles upon freeze-drying, leading to compositions with osmolalities incompatible with subcutaneous (SC) or intramuscular (IM) injections for typical plasmid DNA dosages [32].

The possibility of using dextrans (polysaccharides) as an alternative to disaccharides to stabilize lyophilized 70 kDa (M_{w}) branched PEI/DNA complexes was investigated. Dextran 3 kDa was as effective as sucrose in preserving complex integrity, while reducing the osmolality of the reconstituted solution by approximately 40% [15]. Dextran 3 kDa/sucrose compositions could be concentrated up to tenfold upon rehydration to near isotonicity, providing dosages more suitable for in vivo injections (such as 1 mg/mL), without modification to particle sizes upon concentration, as determined by absence of variation of turbidities measured. However, to reach that final concentration after a tenfold concentration, particles had to be prepared at an initial DNA concentration of 200 μg/mL prior to addition of lyoprotectants [15], which is above the typical maximal concentration (100 μg of DNA per mL) ensuring production of small uniformly sized nanoparticles [1], suggesting that particle sizes and polydispersities in these compositions may have been higher.

Also, the main drawback of using dextrans is their known incompatibility with PEG, their stabilizing effect decreasing with the increasing degree of PEGylation of lipoplexes [17]. Dextran 5 kDa prevented full aggregation of PEGylated PEI/DNA polyplexes, although particle size still increased by 170 to 240% [7]. Inulin, another oligosaccharide, would be an efficient lyoprotectant for PEGylated lipoplexes or polyplexes [7].

More recently, addition of a buffer, 10 mM L-histidine at pH 6, to linear PEI/DNA complexes, prepared at amino/phosphate (N/P) ratio of 6, led to a decrease in particle hydrodynamic diameter (from 176 to 118 nm) and polydispersity index (PDI) (from 0.18 to 0.13), to an increase in zeta potential (from 29.6 to 36.3 mV), but had no significant impact on their in vitro metabolic activity and gene expression [36]. Dextran was found to be a poor lyoprotectant for these complexes, whereas sucrose, at lyoprotectant/DNA weight ratio of at least 2000 (equivalent to 10% (w/v) for compositions containing 50 μg of DNA per mL), stabilized them upon freeze-drying. Isotonic compositions containing 14% lactosucrose, 10% hydroxypropylbetadex/6.5% sucrose or 10% povidone/6.3% sucrose were stable over storage at 40° C. for 6 weeks, with particles smaller than 170 nm. Lactosucrose or hydroxypropylbetadex/sucrose compositions were the most effective in vitro [36]. Another buffer, triethanolamine at pH 7, was found to be effective at preserving PEI-mannobiose (PEIm)/pDNA complexes size upon freeze-dried in combination with 50% glycerol. Freeze-dried compositions could be stored at −20° C. or 4° C. for 30 days, and still preserve particle size at 200 nm (WO 2010/125544) [37].

Using PEI covalently conjugated to polyethylene glycol (PEG) and cholesterol (Chol), PEG-PEI-Chol (0.554 mg/mL)/pDNA(0.15 mg/mL), lipopolyplexes prepared in lactose or sucrose (0.3, 1.5 or 3% (w/v)) could be freeze-dried and rehydrated to final DNA concentrations of 5, 1 or 0.5 mg/ml, without addition of a buffer to the compositions. Little variation in particle size or biological activity (in vitro, in vivo, in cancer patients) was seen between these compositions, after storage of freeze-dried samples for 2 years at −20 or −80° C., in 60% RH, or after reconstitution and storage of samples for up to 3 months at 4° C. (WO 2009/021017) [35]. In fact, lipopolyplexes had been previously reported to be less susceptible to degradation following freeze-thawing in lower sucrose contents (equivalent to 0.0625% (w/v) for compositions containing 50 μg of DNA per mL), with no modifications to their physico-chemical properties and tansfection efficiencies of at least 50% of control [32].

Given the current state of the art, there is still a need for polyelectrolyte complex compositions that provide increased stability of the polyelectrolyte complex in solution and that improve the resistance of the polyelectrolyte complex to physical or chemical degradation over long-term storage involving for example freeze-drying, and be rehydrated at concentrations representing effective doses that are near iso-osmolar.

### SUMMARY OF THE INVENTION

Various aspects of this invention relate to a polyelectrolyte complex composition comprising a polymer, a nucleic acid molecule, a lyoprotectant, and a buffer, said composition preserving biological activities of the polyelectrolyte complex following freeze-drying and rehydration.

Various aspects of this invention relate to a polyelectrolyte complex composition comprising a chitosan, a deoxyribonucleic acid in an amount of about 50 μg/mL, trehalose in an amount of between about 0.5% (w/v) and about 1% (w/v) and histidine in an amount of between about 3 mM and about 4 mM.

Various aspects of this invention relate to a polyelectrolyte complex composition comprising a chitosan, a deoxyribonucleic acid in an amount of about 100 μg/mL, trehalose in an amount of between about 1% (w/v) and 2% (w/v) and histidine in an amount of between about 6 mM and about 8 mM.

Various aspects of this invention relate to a method for preparing a polyelectrolyte complex composition which preserves its biological activities following freeze-drying and rehydration, the method comprising the steps of: mixing chitosan with a lyoprotectant and a buffer forming a chitosan composition; separately mixing a nucleic acid with the lyoprotectant and the buffer forming a nucleic acid composition; and mixing the chitosan composition with the nucleic acid composition to form the polyelectrolyte complex composition.

Various aspects of this invention related to a kit comprising a polyelectrolyte complex composition as defined herein; and instructions for reconstitution of the composition.

Various aspects of this invention related to the use of a polyelectrolyte complex composition as defined herein for delivering a nucleic acid to a subject in need thereof.

### BRIEF DESCRIPTION OF THE FIGURES

Reference will be made to the accompanying drawings.

FIGS. 1A, B and C. FIG. 1A illustrates a graph showing nanoparticle aggregation (5 fold increase in size) seen following freeze-thawing (F/T) in the absence of lyoprotectant, whereas aggregation was prevented (diameter≦150 nm) upon addition of at least 1% w/V mannitol, 0.5% (w/v) sucrose, 0.5% (w/v) dextran 5 kDa, or 0.1% (w/v) trehalose dihydrate to the composition; FIG. 1B illustrates a graph showing transfection efficiency using at minimum the indicated lyoprotectant contents; FIG. 1C illustrates a graph showing luciferase expression of nanoparticles maintained following freeze-thawing, while a significant decrease was seen in the absence of lyoprotectant. Transfection efficiencies and luciferase expression levels were expressed in terms of percentage of the fresh control without excipients (0FT), which had a transfection efficiency of 43% of total cells and a luciferase expression level of 8.03E10 RLU/min·mg of proteins.

FIG. 2A-H. FIG. 2 illustrates images showing nanoparticles freeze-thawed in presence of 1 or 10% (w/v) mannitol (FIG. 2A-2B), sucrose (FIG. 2C-2D) or dextran 5 kDa (FIG. 2E-2F) are more spherical than freshly prepared particle compositions containing no lyoprotectant (FIG. 2G). Nanoparticles freeze-thawed in the absence of lyoprotectant were severely aggregated (FIG. 2H).

FIG. 3A-D. FIG. 3A illustrates a graph showing freeze-drying and rehydration to equal volume of compositions containing of 0.5% (w/v) sucrose, dextran 5k Da, or trehalose dihydrate led to nanoparticle aggregation; rehydrated particles had Z-averages up to 24-fold greater than freshly prepared complexes; FIG. 3B illustrates a graph showing their mean sizes in intensity were up to 9.5-fold greater than freshly prepared particles; FIG. 3C illustrates a graph showing polydispersity indexes (PDI) were above 0.35; and FIG. 3D illustrates a graph showing zeta potentials were null or negative.

FIG. 4A-C. FIG. 4A-C illustrates graphs showing that addition of citric acid/trisodium citrate buffer at pH 4.5 or pH 6.5 to compositions containing 0.5% lyoprotectant led to formation of microscopic aggregates in fresh samples and to total aggregation post freeze-thawing.

FIG. 5A-B. FIG. 5A illustrates an image showing that freshly prepared chitosan/DNA complexes have different morphologies; FIG. 5B illustrates an image showing that addition of L-histidine pH 6.5, to reach a final concentration of 13.75 mM, leads to the formation of slightly larger, more spherical nanoparticles.

FIG. 6A-C. FIG. 6A-B illustrates graphs showing that addition of L-histidine to compositions containing 0.5% lyoprotectant caused no aggregation: a slight increase in particle sizes is seen in fresh and freeze-thawed compositions; FIG. 6C illustrates a graph showing that PDI remained inferior to 0.35.

FIG. 7A-I. FIG. 7A-C illustrates graphs showing nanoparticles freeze-dried in the presence of 0.5 or 1% (w/v) excipient and 13.75 mM histidine could be rehydrated with as low as 10% of their original volume without affecting particle size, although their PDI decreased; FIG. 7D-F illustrates graphs showing that composition containing 0.5% (w/v) sucrose or trehalose dihydrate, and 13.75 mM histidine, could be freeze-dried and rehydrated to the original volume (Rh1×) or to 20 times (Rh20×) their initial concentration without particle aggregation; FIG. 7G-I illustrates graphs showing that 0.5% (w/v) sucrose or trehalose dihydrate compositions could be freeze-dried with as little as 3.44 mM L-histidine without changes to their particle size or PDI.

FIG. 8A-GH. FIG. 8A-C illustrates graphs showing that dilution of chitosan and DNA with excipients prior to complex formation had little impact on size of fresh nanoparticles, though presence of L-histidine yielded particles with lower PDIs, FIG. 8D-H illustrates graphs showing that no particle size changes were seen (Z-average or mean size in intensity) upon Rh1× or Rh20× of compositions containing 0.5% (w/v) sucrose or trehalose and 3.44 mM histidine, though PDIs were slightly lower at Rh20×. With 0.5% (w/v) dextran, particles were larger, but remained around 300 nm, and PDI decreased from 0.4, at Rh1×, to 0.18, at Rh20×.

FIG. 9A-D. FIG. 9A-B illustrates graphs showing transfection efficiencies and luciferase expression levels were expressed in terms of percentage of the fresh control without excipients (No Lyo-His(0)-Fresh), which had a transfection efficiency of 53% of total cells and expression level of 6.76E-5 μM of luciferase/mg of proteins. Compositions containing 0.5% (w/v) lyoprotectant had transfection efficiencies near 100% of control prior to freeze-drying, and below 45% of control post freeze-drying, whereas their luciferase expression levels post freeze-drying were less than 25% of control. FIG. 9C-D illustrates graphs showing that compositions containing both 0.5% lyoprotectant and 3.44 mM L-histidine had transfection efficiencies near 110% of control prior freeze-drying, above 80% of control after rehydration to equal volume (Rh1×), and up to 77% of control after rehydration at 20× for compositions containing trehalose. Compositions containing L-histidine and sucrose or trehalose dihydrate had luciferase expression levels similar to the control (116 to 66% of control), whereas expression was 57 to 12% of control for those containing dextran 5 kDa.

FIG. 10A-F. FIG. 10A-D illustrates graphs showing that concentrating nanoparticle compositions containing 0.5% trehalose dihydrate and 3.5 mM L-histidine by a factor of 20× upon rehydration led to a small increase in size and PDI, but no change in zeta potential of complexes; using one or two successive freeze-drying/rehydration cycles to reaching the final 20× concentration factor had no impact on nanoparticle physico-chemical properties; FIG. 10E-F illustrates graphs showing that compositions concentrated 20× after a single freeze-drying (FD/Rh20×) had a transfection efficiency of 100% of control, whereas after two successive freeze-drying cycles [Rh(10×+2×) and Rh(5×+4×)], they had transfection efficiencies of at least 85% of control. All compositions with final concentration factor of 20× had a luciferase expression level of 64 to 69% of control, whereas they were freeze-dried once or twice.

FIG. 11A-C. FIG. 11A-B illustrates graphs showing that minimal particle size changes were seen (Z-average) upon Rh1× or Rh20× of CS/siRNA compositions containing 1 or 2% (w/v) trehalose and 7 or 3.5 mM histidine, for an siRNA concentration of 100 μg/mL after particle formation. PDIs of these compositions were below 0.25 after Rh1× and below 0.20 after Rh20×; FIGS. 11C illustrates a graph showing that the composition containing 1% trehalose and 7 mM L-histidine preserved silencing efficiency after FD, with residual eGFP expression levels between 52 and 47% of untreated cells, whether compositions were fresh, Rh1× or Rh10×.

### DETAILED DESCRIPTION OF EMBODIMENTS

The present invention stems from the discovery by the inventors that chitosan nucleic acid nanoparticles can be freeze dried and concentrated upon rehydration without changes in particle size or loss of biological activity or creation of hyperosmotic solutions, provided that appropriate lyoprotectant type and concentration, and buffer type and concentration, are present in the particle suspension to be lyophilized.

As such, one embodiment of the present invention provides for polyelectrolyte complex compositions that provide increased stability of the polyelectrolyte complex in solution and/or improved resistance of the polyelectrolyte complex to physical or chemical degradation over long-term storage.

Another embodiment of the present invention provides for freeze-dried polyelectrolyte complex compositions that provide stability of the polyelectrolyte complex in solution and/or improved resistance of the polyelectrolyte complex to physical or chemical degradation over long-term storage.

In some of these implementations, the polyelectrolyte complex is a polysaccharide based polyelectrolyte complex. In some instances, the polyelectrolyte complex is a polyelectrolyte complex between a polysaccharide and a nucleic acid.

As used herein, the term “polyelectrolyte” refers to polymers whose repeating units bear an electrolyte group. As such, polyelectrolytes include polycations and polyanions. These groups dissociate in aqueous solutions, making the polymers charged. Polyelectrolyte properties are thus similar to both electrolytes and polymers.

As used herein, the term “polysaccharide” refers to molecules composed of long chains of monosaccharide units bound together by glycosidic linkages and on hydrolysis give the constituent monosaccharides or oligosaccharides.

In some instances, the polysaccharide is chitosan. As used herein, the term “chitosan” refers to a linear polysaccharide composed of randomly distributed β-(1-4)-linked D-glucosamine (deacetylated unit) and N-acetyl-D-glucosamine (acetylated unit). It is typically made by treating shrimp and other crustacean shells with the alkali sodium hydroxide. Chitosan possesses a wide range of beneficial properties including biocompatibility, biodegradability, mucoaadhesive properties, antimicrobial/antifungal activity and very low toxicity.

The molecular weight of chitosan as well as the amount of amine groups (degree of deacetylation or DDA) on the chain have a major influence on its biological and physiological properties. For example, the amount and distribution of acetyl groups affects biodegradability since the absence of acetyl groups or their homogeneous distribution (random rather than block) results in very low rates of enzymatic degradation.

In some implementations of these embodiments, chitosan may comprise chemical modifications. Examples of chitosan comprising chemical modification include, but are not limited to: chitosan-based compounds having: (i) specific or non-specific cell targeting moieties that can be covalently attached to chitin and/or chitosan, or ionically or hydrophobically adhered to a chitosan-based compound complexed with a nucleic acid or an oligonucleotide, and (ii) various derivatives or modifications of chitin and chitosan which serve to alter their physical, chemical, or physiological properties. Examples of such modified chitosan are chitosan-based compounds having specific or non-specific targeting ligands, membrane permeabilization agents, sub-cellular localization components, endosomolytic (lytic) agents, nuclear localization signals, colloidal stabilization agents, agents to promote long circulation half-lives in blood, and chemical derivatives such as salts, O-acetylated and N-acetylated derivatives. Some sites for chemical modification of chitosan include: C₂(NH—CO—CH₃ or NH₂), C₃(OH), or C₆(CH₂OH).

In some implementations of the embodiments defined herein, chitosan has a specific average molecular weight (Mn) that is between about 4 kDa to about 200 kDa, preferably between about 5 kDa and about 200 kDa, more preferably between about 5 kDa and about 100 kDa, more preferably between about 10 kDa and about 80 kDa. The chitosan further has a specific degree of deacetylation (DDA) that is preferably between about 70% and about 100%, more preferably between about 80% and 95%.

In some implementations of these embodiments, the nucleic acid is one or more of deoxyribonucleic acid (DNA) and ribonucleic acid (RNA). The nucleic acid is for example, one or more of a plasmid (pDNA), a minicircle or an oligodeoxynucleotide (ODN). The nucleic acid may also be one or more of a small interfering ribonucleic acid (siRNA) and a small hairpin ribonucleic acid (shRNA) or a messenger ribonucleic acid (mRNA).

The ratio of polymer and nucleic acid entering into the compositions defined herein is determined in terms of the ratio of moles of the amine groups of polymers to those of the phosphate ones of the nucleic acid (N/P ratio). In some implementations, the N/P ratio of the compositions defined herein is between about 1.2 and about 30, preferably the N/P ratio is between about 2 and about 10, more preferably the N/P ratio is about 5.

Studies with chitosan/nucleic acid polyeletrolyte complexes have been performed [38-45]. These studies involved compositions for high efficiency transfection containing a nucleic acid (such as for example, DNA or RNA) and a chitosan with number-average molecular weight (Mₙ) between 8 and 200 kDa and degree of deacetylation (DDA) between 72% and 95% (WO 2009/0075383 and WO 2012/159215). However, these studies had neither considered nor addressed the multiple challenges related with long-term stabilization of these compositions.

Prior work had also not addressed the possibility of producing isotonic chitosan/nucleic acid compositions at therapeutic concentrations through rehydration of freeze-dried compositions in reduced volumes. Disaccharides (such as sucrose or mannitol) would prevent aggregation and loss of functionality of chitosan-based polyplexes upon freeze-drying and short-term storage (. 2 months) [8-10]. The initial composition pH would be critical to the state of chitosan hydrolysis during freeze-drying, degradation rate increasing 30 fold upon reduction of the solution pH from 6 to 4.1 [2]. To ensure sufficient buffering of the composition, buffer molar concentration must be at least equal to that of chitosan monomer, yet be below 0.1M in the final compositions injected to prevent competition with physiological buffers, and other undesirable effects. The chitosan hydrolysis rate would increase with increasing HCl concentration [18], and would decrease in the presence of solvents promoting more compact chitosan chain conformations, glycosidic bonds located in the center of the structure being less accessible for hydrolysis [19].

Retinol was encapsulated in water-soluble chitosan (18 kDa, 96% DDA) to form spherical nanoparticles that were subsequently lyophilized for 3 days and readily rehydrated in absence of any lyoprotectants. While these rehydrated particles had slightly smaller mean sizes and distribution broadness, lyophilization had no impact on their zeta potential nor degraded the encapsulated retinol [46]. Freeze-dried chitosan (80 kDa, 85% DDA)/poly(y-glutamic acid) nanoparticles for oral insulin delivery were freeze-dried in 1.5% trehalose without modification to size or morphology of rehydrated complexes (mean size ≦245 nm, PDI<0.3) or degradation of the insulin content, despite strong collapse of dry cakes [47]. Chitosan nanoparticles were shown to be sensitive to hydrolysis at acidic pH values: at pH=1.2, particles were degraded; at pH=2.0, they were 28% larger. Polyelectrolyte complexes formed with trimethyl chitosan (TMC; 200 kDa, 85% DDA, degree of quaternization (DQ) of 15 or 30%), or TMC-cysteine conjugates (TMC-Cys), and insulin, were lyophilized in sucrose, at sucrose/insulin w/w ratio of 20, without modification to particle size, zeta potential, or insulin encapsulation efficiency following rehydration [48]. Alginate (75-100 kDa)/chitosan (65-90 kDa, DDA>80%) nanoparticles for delivery of gatifloxacin were formulated in 5% w/v mannitol, freeze-dried and stored at room temperature for up to 12 months. Following rehydration at initial volume, only minimal increase in particle size was noted (from 345 to 410 nm), with no change to their zeta potential or their in vitro gatifloxacin release profile [49]. Methotrexate-incorporated polymeric nanoparticles of methoxy poly(ethyleneglycol)-grafted chitosan (10 kDa, 97% DDA) copolymer were prepared, lyophilized in absence of lyoprotectant for two days, rehydrated in deionized water, and then characterized: particle sizes were below 100 nm, zeta potential values ranged from +20 to +40 mV, and loading efficiency was above 65% [50].

As used herein, the expression “Zeta potentials” refer to electrokinetic potential in colloidal systems. It is typically denoted using the Greek letter zeta (ζ), hence ζ-potential. The zeta potential is the electric potential in the interfacial double layer (DL) at the location of the slipping plane versus a point in the bulk fluid away from the interface. Zeta potential is the potential difference between the dispersion medium and the stationary layer of fluid attached to the dispersed particle.

Nanoparticles of chitosan and polyglutamic acid (PGA), alpha-PGA, soluble salts of PGA, metal salts of PGA or heparin were produced for delivery of nucleic acid to target sites for treatment of osteoporosis. Nanoparticles had an average size of 266 nm, and could be freeze-dried and rehydrated in as low as 2.5% trehalose, with a size increase of 13%, or 2.5% mannitol, with average particle size increasing by 57% (U.S. Pat. No. 7,901,711) [51]. PEGylated chitosan (110 kDa, 87% DDA)/pDNA nanoparticle were lyophilized in 1% mannitol, and then stored 1 month at 4° C. or −20° C., or lyophilized in 40% sucrose, and then stored at −20° C., without any changes to their size, zeta potential and transfection efficiency [8]. Other nanoparticles, DNA:Chitosan (90 kDa):Lytic Peptide (charge ratio 1:6:1 −/+/−), had their size preserved (300-350 nm) upon freeze-drying in 10% lactose, and in vivo expression of the reporter gene CMV-CAT was shown in rabbits 72 hours after oral administration (WO 97/42975) [52].

Investigation of lyophilization of chitosan (CS: 170 kDa, 84% DDA)/siRNA complexes (N/P ratio of 50) showed that 10% sucrose was necessary to preserve particle size after rehydration. Particle size slightly increased in fresh compositions following addition sucrose (126 to 169 nm) and was 142 nm after freeze-drying and rehydration [9]. In absence of lyoprotectant, rehydrated complexes were too large for size measurement in dynamic light scattering (DLS). Sample gene knockdown efficiency increased with the siRNA concentration and was dependent on the presence of sucrose: for lower siRNA concentrations 25 nM), highest knockdown (60%) was obtained with 10% sucrose; for the highest siRNA concentration (50 nM), 5% sucrose was sufficient to reach maximum knock down efficiency (70%). A 10% H1299 cell viability decrease was measured when formulating chitosan/siRNA (50 nM) complexes in 5% sucrose or more. Silencing activity of 10% sucrose compositions reached 32% after storage for 2 months at room temperature [9].

Chitosan coated PLGA complexes used for delivery of oligonucleotides and siRNA where shown to aggregate when freeze-dried in a solution of 0.05% (w/v) chitosan and 1% (w/v) polyvinylalcohol (PVA) [10]. Complex aggregation also resulted upon freeze-drying in a more concentrated composition supplemented with a buffer: 0.25% (w/v) chitosan, 10% (w/v) PVA, and 0.5M acetate buffer at pH4.4 [53]. Aggregation upon freeze-drying could be avoided by addition of mannitol at a lyoprotectant:nanosphere weight ratio greater than 5:1 [10]. In absence of chitosan coating, aggregation of PLGA/oligonucleotide particles upon freeze-drying could be avoided by using a lyoprotectant:nanosphere weight ratio greater than 1:1 only [10].

Finally, chitosan/DNA complexes were formed in Tris-HCl buffer, isolated by centrifugation in aqueous medium, and filled into molds prior to freeze-drying in absence of lyoprotectant (JP 4354445) [54].

In order to preserve physico-chemical properties and transfection efficiencies of polymer/nucleic acid polyplexes prepared in dilute regime upon freeze-drying, compositions need to include concentrations of lyoprotectants (disaccharides, trisaccharides, or polyols) which are incompatible with rehydration to isotonic injections at 0.5 to 1 mg of DNA per mL. Final dosages of injections are therefore highly limited since freeze-dried compositions cannot be rehydrated to higher concentrations without being highly hypertonic. Addition of a buffer to high concentrations of lyoprotectant has little effect on preservation of nanoparticle properties post-lyophilization. However, its presence may be required to control the pH of rehydrated compositions with lower lyoprotectant concentrations prior to injection. Although dextran/sucrose compositions allow rehydration of freeze-dried branched PEI-based compositions to near isotonicity upon a tenfold concentration, these compositions are limited to dextrans and sucrose, and do not include any buffer which may be necessary for maintenance of particle size and integrity post-lyophilization.

In another embodiment, the polyelectrolyte complex compositions comprise a polymer, a nucleic acid molecule and a freeze-drying protectant. As used herein, the expression “freeze-dry protectants” refers to molecules that protect freeze-dried materials. Freeze-dry protectants includes, for example, cryoprotectants and lyoprotectants. Known lyoprotectants include, but are not limited to, polyhydroxy compounds such as sugars (mono-, di-, and polysaccharides), polyalcohols, and their derivatives. Trehalose and sucrose are natural lyoprotectants. Trehalose is produced by a variety of plant (for example selaginella and arabidopsis thaliana), fungi, and invertebrate animals that remain in a state of suspended animation during periods of drought (also known as anhydrobiosis). In some implementations of this embodiment, the lyoprotectant is one or more of a disaccharide, a trisaccharide, an oligosaccharide/polysaccharide, a polyol, a polymer, a high molecular weight excipient, an amino acid molecule or any combination thereof. The disaccharide may be one or more of sucrose, trehalose, lactose, maltose, cellobiose, and melibiose. The disaccharide may be present in the compositions of the invention at a concentration that is between about 0.1% (w/v) and about 10% (w/v), preferably between about 0.5% (w/v) and about 5% (w/v), and more preferably between about 0.5% (w/v) and about 2% (w/v). The trisaccharide may be one or more of maltotriose and raffinose. The trisaccharide may be present in a concentration of between about 0.1% (w/v) and about 10% (w/v), preferably between about 0.5% (w/v) and about 5% (w/v), and more preferably between about 0.5% (w/v) and about 2% (w/v). The oligosaccharide/polysaccharide may be one or more of dextran, cyclodextrin, maltodextrin, hydroxyethyl starch, ficoll, cellulose, hydroxypropylmethyl cellulose, and inulin. The oligosaccharide/polysaccharide may be present in the compositions of the invention at a concentration that is between about 0.1% (w/v) and about 10% (w/v), preferably between about 0.5% (w/v) and about 5% (w/v), and more preferably between about 0.5% (w/v) and about 2% (w/v). The dextran may be useful for applying osmotic pressure to biological molecules. In some implementations, the dextran has an average molecular weight (Mₙ) of between 1 and 70 kDa, preferably of between 1 and 5 kDa. The polyol may be one or more of mannitol and inositol. The polyol may be present in the compositions of the invention at a concentration that is between about 0.1% (w/v) and about 10% (w/v), preferably between about 0.5% (w/v) and about 5% (w/v), and more preferably between about 2% (w/v) and about 3% (w/v). The amino acid molecule may be at least one of lysine, arginine, glycine, alanine and phenylalanine. The amino acid molecule may be present in the compositions of the invention at a concentration that is between about 1 mM and about 100 mM, preferably between about 3 mM and about 14 mM, and more preferably between about 3 mM and about 4 mM. The high molecular weight excipient may be one or more of polyethylene glycol (PEG), gelatin, polydextrose and polyvinylpyrrolidone (PVP).

In some other embodiments, the polyelectrolyte complex compositions comprise a polymer, a nucleic acid molecule, a freeze-drying protectant and a buffer. The buffer for the present compositions may comprise at least one of sodium citrate, histidine, sodium malate, sodium tartrate and sodium bicarbonate. The buffer may be present in the composition defined herein at a concentration that is between about 1 mM and about 100 mM, preferably between about 3 mM and about 14 mM, preferably between about 3 mM and about 8 mM, and more preferably between about 3 mM and about 4 mM.

In some instances of these embodiments, the polyelectrolyte complex composition comprises a polymer, a nucleic acid, trehalose and histidine.

In some instances of these embodiments, the polyelectrolyte complex composition comprises a chitosan, a nucleic acid, trehalose and histidine.

In other instances of these embodiments, the polyelectrolyte complex composition comprises a polymer, a nucleic acid, trehalose in an amount of about 0.5% (w/v) to about 2% (w/v) and histidine in an amount of about 3 mM to about 8 mM.

In other instances of these embodiments, the polyelectrolyte complex composition comprises a chitosan, a nucleic acid, trehalose in an amount of about 0.5% (w/v) to about 2% (w/v) and histidine in an amount of about 3 mM to about 8 mM.

In other instances of these embodiments, the polyelectrolyte complex composition comprises a chitosan, a deoxyribonucleic acid in an amount of about 50 μg/mL, trehalose in an amount of about 0.5% (w/v) to about 1% (w/v) and histidine in an amount of about 3 mM to about 4 mM.

In other instances of these embodiments, the polyelectrolyte complex composition comprises a chitosan, a ribonucleic acid in an amount of about 100 μg/mL, trehalose in an amount of about 1% (w/v) to about 2% (w/v) and histidine in an amount of about 6 mM to about 8 mM.

In other instances, the polyelectrolyte complex compositions comprise a polymer, a nucleic acid, sucrose and histidine.

In other instances, the polyelectrolyte complex compositions comprise a chitosan, a nucleic acid, sucrose and histidine.

In other instances, the polyelectrolyte complex compositions comprise a polymer, a nucleic acid, sucrose in an amount of about 0.5 (w/v) to about 2% (w/v) and histidine in an amount of about 3 mM to about 4 mM.

In other instances, the polyelectrolyte complex compositions comprise a chitosan, a nucleic acid, sucrose in an amount of about 0.5 (w/v) to about 2% (w/v) and histidine in an amount of about 3 mM to about 4 mM.

According to certain implementations of the present embodiments, the polyelectrolyte complex composition is freeze-dried. As will be understood, freeze-drying, also known as lyophilisation, lyophilization, or cryodesiccation, is a dehydration process used to preserve a perishable material or make the material more convenient for transport. Freeze-drying works by freezing the material and then reducing the surrounding pressure to allow the frozen water in the material to sublimate directly from the solid phase to the gas phase.

The process of freeze-drying may involve a pre-treatment step including any method of treating the product prior to freezing. This step may involve actions such as, but not limited to addition of components to increase stability and/or improve processing, decreasing a high vapor pressure solvent or increasing the surface area. Methods of pre-treatment include: freeze concentration, solution phase concentration, formulation to preserve product appearance, formulation to stabilize reactive products, formulation to increase the surface area, and decreasing high vapor pressure solvents.

On a small scale, freezing is typically done by placing the material in a freeze-drying flask and rotating the flask in a bath, called a shell freezer, which is cooled by mechanical refrigeration, dry ice and methanol, or liquid nitrogen. On a larger scale, freezing is usually done using a freeze-drying machine. In this step, it is important to cool the material below its triple point, the lowest temperature at which the solid and liquid phases of the material can coexist. This ensures that sublimation rather than melting will occur in the following steps. Larger crystals are easier to freeze-dry.

During a primary drying phase, the pressure is lowered, and enough heat is supplied to the material for the water to sublime. The amount of heat necessary can be calculated using the sublimating molecules' latent heat of sublimation. In this initial drying phase, about 95% of the water in the material is sublimated. In this phase, pressure is controlled through the application of partial vacuum. The vacuum speeds up the sublimation, making it useful as a deliberate drying process. Furthermore, a cold condenser chamber and/or condenser plates provide a surface(s) for the water vapor to re-solidify on. A secondary drying phase aims to remove unfrozen water molecules, since the ice was removed in the primary drying phase. This part of the freeze-drying process is governed by the material's adsorption isotherms. In this phase, the temperature is raised higher than in the primary drying phase, and can even be above 0° C., to break any physico-chemical interactions that have formed between the water molecules and the frozen material.

Suitable freeze-dryers include but are not limited to a manifold freeze-dryer, the rotary freeze-dryer and the tray style freeze-dryer.

In another embodiment, the present invention also provides methods for preparing the polyelectrolyte complex and polyelectrolyte complex compositions as defined herein. In one implementation of this embodiment, the method comprises preparing a polymer composition and a nucleic acid composition; mixing the polymer and nucleic acid compositions together to form a polyelectrolyte complex composition. The resulting polyelectrolyte complex composition may then be freeze-dried.

In some implementations of this embodiment, the method comprises a step wherein the polymer is dissolved and a step wherein the dissolved polymer is mixed with a suitable freeze-drying protectant and a suitable buffer so as to form a polymer composition. The method also comprises a step wherein the nucleic acid molecule is mixed with a suitable freeze-drying protectant and a suitable buffer so as to form a nucleic acid composition. The polymer and the nucleic acid compositions are then mixed together to form a polyelectrolyte complex composition. The resulting polyelectrolyte complex composition may then be freeze-dried.

In another implementation, the method comprises a step wherein chitosan is dissolved and a step wherein the dissolved chitosan is mixed with a suitable lyoprotectant and a suitable buffer so as to form a chitosan composition. The method also comprises a step wherein the nucleic acid molecule is mixed with a suitable lyoprotectant and a suitable buffer so as to form a nucleic acid composition. The chitosan and the nucleic acid compositions are then mixed together to form a polyelectrolyte complex composition. The resulting polyelectrolyte complex composition

The present invention also provides an article of manufacture or a commercial package or kit, comprising one or more of a container, a label on the container, polyelectrolyte complex compositions as defined herein, and instructions for use. In addition to the polyelectrolyte complex compositions as defined herein, the article of manufacture or commercial package or kit may also comprise water for reconstitution of the polyelectrolyte complex composition prior to use.

The present invention also provides an article of manufacture or a commercial package or kit, comprising one or more of a container, a label on the container, freeze-dried polyelectrolyte complex compositions as defined herein, and instructions for use. In addition to the freeze-dried polyelectrolyte complex compositions as defined herein, the article of manufacture or commercial package or kit may also comprise water for reconstitution of the polyelectrolyte complex composition prior to use. Suitable

In some implementations of this embodiment, the water is suitable for injection into a subject. The polyelectrolyte complex composition may be reconstituted in water at a concentration at 5-fold, 6-fold, 7-fold, 8-fold, 9-fold, 10-fold, 11-fold, 12-fold, 13-fold, 14-fold, 15-fold, 16-fold, 17-fold, 18-fold, 19-fold or 20-fold the initial concentration. The polyelectrolyte complex composition may be reconstituted in water at a concentration of 25-fold, 30-fold, 35-fold, 40-fold, 45-fold, 50-fold, 55-fold, or 60-fold the initial concentration by performing more than one reconstitution cycles, such as for example, by performing 2 reconstitution cycles.

The polyelectrolyte complex compositions as defined herein may be prepared in dilute conditions (such as for example, but not limited to, about 100 μg of nucleic acid per mL, specific to each composition) to produce small uniformly sized nanoparticles for nucleic acid delivery. Freeze-dried and rehydrated compositions may have small nanoparticle sizes and low polydispersity indexes.

As used herein, the term “polydispersity” or “polydispersity index” (PDI) refers to a measure of the distribution of molecular mass in a given polymer sample. The PDI calculated is the weight average molecular weight divided by the number average molecular weight. It indicates the distribution of individual molecular masses in a batch of polymers. The PDI has a value equal to or greater than 1, but as the polymer chains approach uniform chain length, the PDI approaches unity (1).

In some implementations of the embodiments, the freshly prepared, freeze-dried and/or rehydrated compositions as defined herein have one or more of the following properties:
A) They have positive zeta potentials to promote cell uptake during transfection. The zeta potentials being sufficiently high to ensure short-term stability between complex formation and freeze-drying and between composition rehydration and injection.
B) They present uniform lyophilization of nanoparticles such that minimal or no aggregation is detected in the compositions following freeze-drying and rehydration.
C) They preserve polyelectrolyte complex biological activity following freeze-drying and rehydration.

As used herein the expression “biological activity” in reference to the polyelectrolyte complexes as defined herein refers to the biological, cellular or pharmacological abilities of the polyelectrolyte complexes as defined herein, in particular their ability to express protein (transfection efficiency) when a plasmid DNA is delivered and their ability to silence gene expression through RNAi when siRNA is delivered, both without inducing undesirable toxicity or immune responses. These biological activities should preferably be retained along with one or more of the properties A-G.
D) For ease-of-use in the clinic, freeze-dried compositions as defined herein are completely reconstituted within a time limit that is convenient for injection into a subject.
E) In order to reach therapeutic dosages with limited injection volumes upon their use in the clinic, the rehydrated polyelectrolyte complex compositions as defined herein have a maximal nucleic acid concentration.
F) They have minimal amounts of excipients in order to be near-isotonic upon rehydration to higher final nucleic acid concentrations. In particular, rehydrated formulations are near-isotonic to minimize cell damage, patient discomfort or pain, upon injection.
G) Rehydrated polyelectrolyte complex compositions as defined herein have near-neutral pH to minimize cell damage, patient discomfort or pain, upon injection. Particularly, the compositions as defined herein may be slightly acidic in order to prevent polycation or nanoparticle precipitation in the solution.

In some implementations of the embodiments, the fresh, freeze-thawed, and/or freeze-dried and rehydrated compositions as defined herein present one or more of the following nanoparticle physico-chemical properties:
A) The nanoparticle Z-average is below 750 nm, preferably below 500 nm, more preferably below 250 nm. The nanoparticle Z-average may be determined by, for example, DLS.
B) The nanoparticle average polydispersity index (PDI) is at most 0.5, preferably at most 0.35, most preferably at most 0.25. The nanoparticle average PDI may be assessed by, for example, DLS.
C) The nanoparticle average zeta potential is positive and sufficient to ensure short-term stability of the compositions. The nanoparticle average zeta potential may be assessed by, for example, LDV.
D) The compositions of the present invention are substantially free of aggregation. The presence of aggregations may be assessed by, for example, ESEM.

The nanoparticles of the compositions as defined herein also present at least one or more of the following in vitro efficiency criteria:
A) They present a transfection level that is greater than about 10%, preferably greater than about 25%, most preferably greater than about 50% of the transfection level of fresh polyelectrolyte particles without excipients. The transfection level may be assessed by, for example, flow cytometry.
B) They present a luciferase expression level that is greater than 10%, preferably greater than 25%, and most preferably that is greater than 50% of the expression level of fresh CS/DNA particles without excipients. The luciferase expression level may be assessed by, for example, luminometry.
C) They present a silencing efficiency that is greater than 10%, preferably greater than 25%, most preferably greater than 50% of the silencing efficiency of fresh polyelectrolyte particles. The silencing efficiency may be assessed by, for example, flow cytometry.

The compositions defined herein present one or more of the following performance criteria upon rehydration which render them suitable for clinical uses:
A) The freeze-dried cake is completely reconstituted within about 10 minutes, more preferably within about 9 minutes, more preferably within about 8 minutes, more preferably within about 7 minutes, more preferably within about 6 minutes and most preferably within about 5 minutes. The level of reconstitution may be assessed by visual inspection upon reconstitution.
B) The final nucleic acid concentration is at least 0.1 mg/mL, preferably at least 0.2 mg/mL, more preferably at least 0.3 mg/mL, more preferably at least 0.4 mg/mL, and most preferably at least 0.5 mg/mL. The final DNA concentration may be determined from the initial DNA content and the rehydration factor used. In some instances, the final DNA concentration is at least 0.1 mg/mL, preferably at least 0.2 mg/mL, more preferably at least 0.3 mg/mL, more preferably at least 0.4 mg/mL, and most preferably at least 0.5 mg/mL. The final RNA concentration may be determined from the initial DNA content and the rehydration factor used. In some other instances, the final RNA concentration is at least 0.1 mg/mL, preferably at least 0.2 mg/mL, more preferably at least 0.3 mg/mL, more preferably at least 0.4 mg/mL, and most preferably at least 0.5 mg/mL. The final RNA concentration may be determined from the initial RNA content and the rehydration factor used.

C) The rehydrated compositions as defined herein are near iso-osmolality. In some instances, the rehydrated compositions as defined herein have an osmolality that is between about 100 and 750 mOsm, preferably between about 150 and 500 mOsm, and most preferably between about 200 and about 400 mOsm. The osmolality of the rehydrated compositions may be determined with the osmolality model of the compositions.
D) The rehydrated compositions as defined herein have a near-neutral pH. In particular aspects, the rehydrated compositions as defined herein have a pH that is between 5 and 8, more preferably between 5.5 and 7.5, most preferably between 6 and 7. The pH of the rehydrated compositions may be determined using a pH meter.

In some embodiments of the present invention, the compositions as defined herein are used in the treatment of disorders or diseases in a subject, wherein the subject is an animal or a human. As used herein, “treatment” and “treating” include preventing, inhibiting, and alleviating conditions and symptoms associated with disorders or diseases. The treatment may be carried out by administering a therapeutically effective amount of the compositions described herein. In some instances, the compositions as defined herein are suitable for injection into a subject such as an animal or a human. The injection may be intradermal, subcutaneous, intramuscular, intravenous, intraosseous, intraperitoneal, intrathecal, epidural, intracardiac, intraarticular, intracavernous or intravitreal.

In some instances, the compositions as defined herein may be used in gene therapy. As used herein, the expression “gene therapy” refers to the use of a nucleic acid, such as DNA, as a drug to treat disease by delivering therapeutic nucleic acid into cells of a subject. The most common form of gene therapy involves using the nucleic acid that encodes a functional, therapeutic gene to replace a mutated gene. Other forms involve directly correcting a mutation, or using DNA that encodes a therapeutic protein drug (rather than a natural human gene) to provide treatment.

The present description will be more readily understood by referring to the following examples which are given to illustrate the invention rather than to limit its scope.

Experiments and Data Analysis

Preparation of Polyelectrolyte Complex Compositions

Room temperature chitosan (Mₙ, 10 kDa, 92% DDA) was weighed into 4-mL Lab File glass vials and Milli-Q water and HCl 1N were added to each vial. The final chitosan concentration was 5 mg/mL, with HCl final concentration of 28 mM. The vials were placed on a rotator and stirred overnight at room temperature to ensure complete dissolution. The chitosan stock solution was filter sterilized.

Addition of excipients to the compositions was done: 1) after complex formation; or 2) before complex formation, when diluting chitosan and DNA or 3) siRNA stock solutions:
1) Under the laminar flow hood, the stock chitosan solution was diluted to 271 μg/mL with Milli-Q water, and then 100 μL was mixed with 100 μL of plasmid DNA (pEGFPLuc) at 100 μg/mL, in order to form complexes at N/P ratio of 5. Mixing was performed by pipetting the solution up and down approximately 10 times immediately following addition of chitosan. Samples were left to stabilize at room temperature for 30 minutes, and then sample volumes were completed to 400 μL with Milli-Q water; and/or sterile 2, 4 or 20% (w/v) mannitol, sucrose, dextran 5 kDa, or trehalose dihydrate; and/or 70 mM citric acid/trisodium citrate buffer at pH 4.5 or 6.5; or 13.75, 27.5 or 55 mM L-histidine at pH 6.5, as required.
2) Under the laminar flow hood, the stock chitosan solution was diluted to 271 μg/ml with Milli-Q water; and/or sterile 2, 4 or 20% (w/v) sucrose, dextran 5 kDa, or trehalose dihydrate; and/or 13.75 or 55 mM L-histidine at pH 6.5, as required. A 200 μg/mL DNA stock solution was diluted to 100 μg/mL following the same method. Then, 100 μL of chitosan composition was mixed with 100 μL of DNA composition, in order to form complexes at N/P ratio of 5. Mixing was performed by pipetting the solution up and down approximately 10 times immediately following addition of chitosan. Samples were left to stabilize at room temperature for 30 minutes.
3) Under the laminar flow hood, the stock chitosan solution was diluted to 271 or 542 μg/ml with RNase-free water; sterile 8% (w/v) dextran 5 kDa and/or trehalose dihydrate; and 14 mM L-histidine at pH 6.5, as required. A 1 mg/mL siRNA stock solution was diluted to 100 or 200 μg/mL following the same method. Then, 100 μL of chitosan composition was mixed with 100 μL of siRNA composition, in order to form complexes at N/P ratio of 5. Mixing was performed by pipetting the solution up and down approximately 10 times immediately following addition of chitosan. Samples were left to stabilize at room temperature for 30 minutes.

Samples to be freeze-thawed were transferred to 1.5 mL cryovials and frozen to −80° C. at a rate of −1° C./min, for at least 2 hours. Samples were thawed at room temperature for 30 minutes prior to use.

Samples to be freeze-dried were transferred to 2 mL serum vials and freeze-dried with 13 mm butyl lyophilization stoppers and a water permeable membrane was placed over the tray containing all samples to prevent dust or bacterial contamination. Freeze-drying was carried in a Millrock Laboratory Series Freeze-Dryer PC/PLC, using one of two cycles:
1) Ramped freezing from room temperature to −40° C. in 1 hour, then maintaining isothermal at −40° C. for 2 hours; primary drying for 48 hours at −40° C., at 100 millitorrs; and secondary drying at 100 millitorrs, increasing temperature to 30° C. in 12 hours and then maintaining isothermal at 30° C. for 6 hours.
2) Step cool to 5° C. and maintain isothermal for 30 min, step cool to −5° C. and maintain isothermal for 30 min, then ramp freeze to −40° C. in 35 min and maintain isothermal for 2 h; primary drying for 48 hours at −40° C., at 100 millitorrs; and secondary drying at 100 millitorrs, increasing temperature to 30° C. in 12 hours and then maintaining isothermal at 30° C. for 6 hours.

Samples were stoppered, crimped and stored at 4° C. until use. 15 to 30 minutes prior to use, samples were rehydrated using a volume of Milli-Q water equivalent to 100%, 20%, 10% or 5% of their original volume, as required.

Particle size and polydipsersity (PDI) was measured by Dynamic Light Scattering (DLS) on 40 μL or 400 μL samples. Whole samples or fractions of samples, diluted with Milli-Q water or excipients, were used for measurements. Diluent viscosity was adjusted in the instrument according to the type of excipients and their final concentration during size analysis. For each sample, at least two consecutive size analyses were done at 25° C., each analysis resulting from 12 to 20 successive readings (10 seconds photon counts/reading) averaged to obtain a data set. The number of successive readings required for each analysis was optimized by the apparatus. Z-average diameter, size distributions by intensity, and PDI were derived from the correlation functions.

Particle zeta potential, or surface charge, was measured by Laser Doppler Velocimetry. Whole samples were diluted with Milli-Q water and NaCl solution to have 800 μL sample with 10 mM NaCl. Diluent viscosity was adjusted in the instrument according to the type of excipients and their final concentration. For each sample, three consecutive zeta potential analyses were done at 25° C., each analysis resulting from 10 to 20 successive readings averaged to obtain a data set. The number of successive readings required for each analysis was optimized by the apparatus.

Nanoparticle morphology was assessed by Environmental Scanning Electron Microscope (ESEM) imaging. Small volumes of sample were pulverized on polished silicon wafers using a gas spray method, and then were sputter-coated with gold. Observations were performed using the high vacuum mode of the ESEM for greater resolution. The high vacuum observation parameters were as follows: accelerating voltage=20 kV; spot size=3; working distance ˜5 mm.

The pH of the different compositions was measured using a microelectrode requiring at least 100 μL of sample to get a measurement.

Given the large volumes of samples involved in measuring osmolality of freeze-dried samples rehydrated at 20-fold their initial concentration, and the apparent negligible impact of nanoparticles on osmolality of fresh solutions, a model was developed to estimate composition osmolalities using excipients only. The osmolality of serial dilutions of sucrose, dextran 5 kDa, trehalose dihydrate and L-histidine were used to establish an additive model, which was then validated with a composition containing 5% (w/v) dextran, 5% (w/v) trehalose dihydrate, and 35 mM L-histidine at pH 6.5. Precision of the model in estimating compositions with nanoparticles at 50 μg of DNA/mL was then verified with compositions containing 3.44 mM histidine at pH 6.5 and 0.5% (w/v) sucrose, dextran or trehalose, freeze-dried and rehydrated 5 times more concentrated. The model was acceptable for the above compositions containing sucrose or trehalose, with osmolality underestimations of 6 and 8% respectively, but was inadequate for dextran containing compositions, with an underestimation of the osmolality of 57%.

Transfection efficiency and gene expression levels of compositions were assessed using a Human Embryonic Kidney 293 (HEK293) cells. HEK293 cells were grown in DMEM high glucose at pH 7.4, supplemented with 10% fetal bovine serum (FBS), and incubated at 37° C. in 5% CO₂. 24 hours prior to transfection, 60,000 cells/well were plated in a 24-well plate, in order to reach about 50% confluency (about 150000 cells per well) on the day of transfection. Each sample was used to transfect two wells of the 24-well plates: one for analysis of transfection efficiency in flow cytometry, the other for luciferase expression quantification. In each well, the exact volume of nanoparticle composition was added, along with transfection medium (DMEM high glucose at pH 6.5 supplemented with 10% FBS), in order to have a total 500 μL of transfection medium and sample containing 2.5 μg of DNA. Cells were then incubated 24 hours at 37° C., in 5% CO₂, and then transfection medium was replaced with 500 μL of growth medium. Cells were incubated an additional 24 hours at 37° C., in 5% CO₂, prior to analysis.

Transfection efficiency was measured in flow cytometry. 20,000 events were collected per sample, and fluorescence was detected through 510/20 nm bandpass filter with photomultiplier tubes following excitation of enhanced green fluorescence protein (EGFP) in transfected cells using a 488 nm argon laser. HEK293 cell line auto-fluorescence was measured using non transfected cells, and fluorescence detection gates were adjusted accordingly. Forward scatter (FSC) and side scatter (SSC) were also used to exclude dead cells and debris from events recorded. Finally, FSC was used to identify and exclude doublets from the events when analyzing transfection efficiency using the data.

Gene expression was assessed by quantifying luciferase proteins content in samples using the Bright-Glo™ Luciferase Assay, and was normalized over the total protein content of each sample, as measured with the Bicinchoninic acid (BCA) assay. Growth medium was removed from each well containing a transfected sample to be analyzed; cells were washed twice with 100 μL PBS at pH 7.4; cells were lysed 5 minutes at room temperature using 100 μL Glo Lysis Buffer per well; and then cell lysates were stored at −20° C. until analysis. Lysates were thawed at room temperature. Luciferase expression was measured in white 96-well plates: 25 μL of Bright-Glo™ Luciferase Reagent was mixed with 25 μL of cell lysate, and then luminescence was measured. Luciferase content was expressed in relative light units per minute (RLU/min) or was converted to pg using a standard curve made of serial dilutions of a recombinant luciferase standard of know concentration. Protein content was measured in clear 96-well plates: 200 μL of BCA working reagent was mixed with 25 μL of cell lysate; samples were incubated 30 minutes at 37° C., 5% CO₂, and then cooled to room temperature; absorbance at 562 nm was measured. A standard curve, prepared using serial dilutions of a 200 μg/mL bovine serum albumin (BSA) standard, was prepared and analyzed alongside samples to convert absorbance readings to protein concentrations.

Silencing efficiency of compositions was assessed using enhanced green fluorescence protein positive human non-small cell lung carcinoma (eGFP positive H1299) cells. Cells were grown in RPMI-1640 at pH 7.4, supplemented with 10% fetal bovine serum (FBS), and incubated at 37° C. in 5% CO₂. 24 hours prior to transfection, 45,000 cells/well were plated in a 24-well plate, in order to reach about 75-85% confluency on the day of transfection. In each well, the exact volume of nanoparticle composition was added, along with DMEM high glucose at pH 6.5 (no FBS), in order to have a total 500 μL of medium and sample containing 100 nM of siRNA. Cells were then incubated 4 hours at 37° C., in 5% CO₂, supplemented with 55 μL FBS, and then incubated an additional 44 hours at 37° C., in 5% CO₂, prior to analysis. Silencing efficiency was measured by flow cytometry. 10,000 events were collected per sample, and fluorescence was detected through a 510/20 nm bandpass filter with photomultiplier tubes following excitation of enhanced green fluorescence protein (EGFP) in transfected cells using a 488 nm argon laser. The mean decrease in eGFP intensity relative to non-treated cells was calculated. Forward scatter (FSC) and side scatter (SSC) were also used to exclude dead cells and debris from events recorded. Finally, FSC was used to identify and exclude doublets from the events when analyzing transfection efficiency using the data.

### EXAMPLES

### Example 1

Lyoprotectants Prevent Nanoparticle Aggregation and Preserve Transfection Efficiency After a Freeze-Thaw Cycle

1—Preparation of Chitosan/DNA Nanoparticle Compositions

Chitosan (Mn 10 kDa, 92% DDA) was dissolved in HCl overnight at room temperature to obtain a final chitosan concentration of 5 mg/mL. The stock solution was diluted to 271 μg/ml, and then 100 μL was mixed with 100 μL of plasmid DNA (pEGFPLuc) at 100 μg/mL in order to form complexes at N/P ratio of 5. Mixing was performed by pipetting the solution up and down approximately 10 times immediately following addition of chitosan. Samples were left to stabilize at room temperature for 30 minutes, and then sample volumes were completed to 400 μL with sterile Milli-Q water and/or sterile 20% (w/v) mannitol, 20% (w/v) sucrose, 20% (w/v) dextran 5 kDa, or 20% (w/v) trehalose dihydrate, as per Table 1.

TABLE 1
Compositions containing lyoprotectants to be analyzed prior to and after freeze-thawing

| Composition | | | Volume added in each sample (μL) | | | | | | |
|---|---|---|---|---|---|---|---|---|---|
| | Lyoprotectant | | Chitosan | DNA | Man | Suc | Dex | Tre | |
| | | % | 271 | 100 | 20% | 20% | 20% | 20% | Milli-Q |
| # | Type | m/V | μg/mL | μg/mL | (w/v) | (w/v) | (w/v) | (w/v) | water |
| 1 | None | 0 | 100 | 100 | 0 | 0 | 0 | 0 | 200 |
| 2 | Mannitol | 0.1 | 100 | 100 | 2 | 0 | 0 | 0 | 198 |
| 3 | (man) | 0.5 | 100 | 100 | 10 | 0 | 0 | 0 | 190 |
| 4 | | 1 | 100 | 100 | 20 | 0 | 0 | 0 | 180 |
| 5 | | 3 | 100 | 100 | 60 | 0 | 0 | 0 | 140 |
| 6 | | 5 | 100 | 100 | 100 | 0 | 0 | 0 | 100 |
| 7 | | 10 | 100 | 100 | 200 | 0 | 0 | 0 | 0 |
| 8 | Sucrose | 0.1 | 100 | 100 | 0 | 2 | 0 | 0 | 198 |
| 9 | (suc) | 0.5 | 100 | 100 | 0 | 10 | 0 | 0 | 190 |
| 10 | | 1 | 100 | 100 | 0 | 20 | 0 | 0 | 180 |
| 11 | | 3 | 100 | 100 | 0 | 60 | 0 | 0 | 140 |
| 12 | | 5 | 100 | 100 | 0 | 100 | 0 | 0 | 100 |
| 13 | | 10 | 100 | 100 | 0 | 200 | 0 | 0 | 0 |
| 14 | Dextran 5 | 0.1 | 100 | 100 | 0 | 0 | 2 | 0 | 198 |
| 15 | (dex) | 0.5 | 100 | 100 | 0 | 0 | 10 | 0 | 190 |
| 16 | | 1 | 100 | 100 | 0 | 0 | 20 | 0 | 180 |
| 17 | | 3 | 100 | 100 | 0 | 0 | 60 | 0 | 140 |
| 18 | | 5 | 100 | 100 | 0 | 0 | 100 | 0 | 100 |
| 19 | | 10 | 100 | 100 | 0 | 0 | 200 | 0 | 0 |
| 20 | Trehalose | 0.1 | 100 | 100 | 0 | 0 | 0 | 2 | 198 |
| 21 | (tre) | 0.5 | 100 | 100 | 0 | 0 | 0 | 10 | 190 |
| 22 | | 1 | 100 | 100 | 0 | 0 | 0 | 20 | 180 |
| 23 | | 3 | 100 | 100 | 0 | 0 | 0 | 60 | 140 |

2—Sample Freeze-Thawing (Without Drying)

Samples to be freeze-thawed were transferred to 1.5 mL cryovials and frozen to −80° C. at a rate of −1° C./min, for at least 2 hours. Samples were thawed at room temperature for 30 minutes prior to use.

3—DLS Measurements

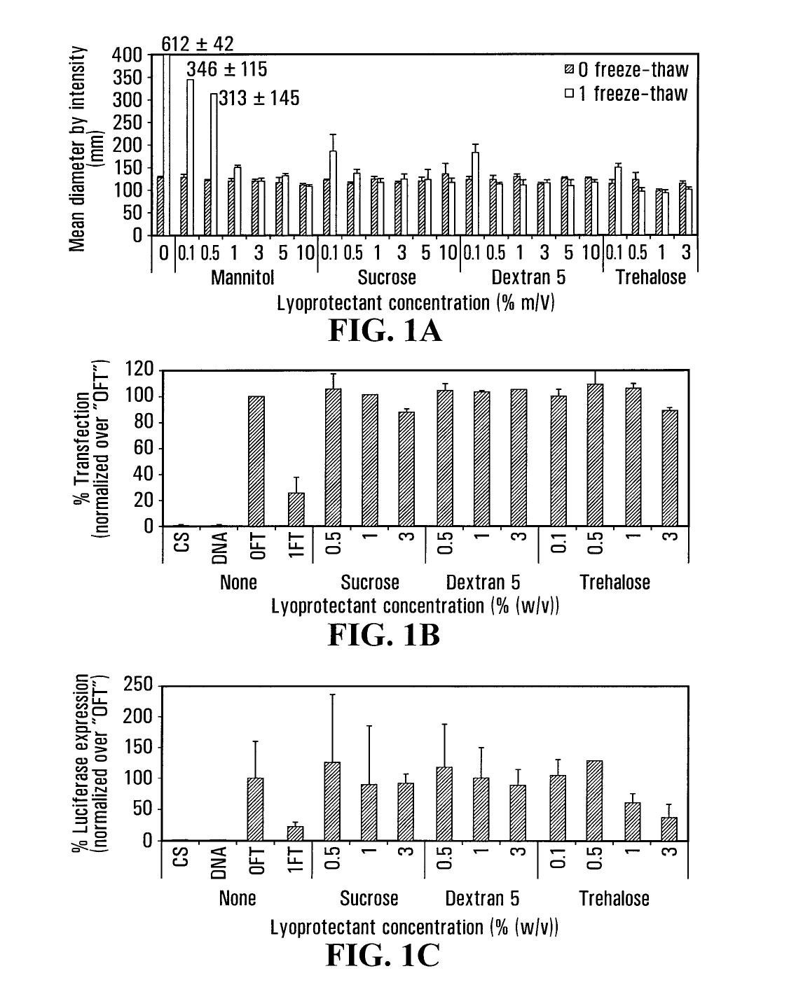

Lyoprotectant screening was performed starting with mannitol, sucrose, and dextran 5 kDa, at concentrations ranging from 0.1% to 10% (w/v). Trehalose dihydrate was later tested, but at concentration ranging from 0.1 to 3% (w/v) only, since particle sizes measured for the first three lyoprotectants remained unchanged above that upper limit, therefore adding more lyoprotectant during screening was deemed unnecessary. Four samples were analyzed per composition: two freshly prepared and two following a freeze-thaw cycle. For each sample, two consecutive DLS size analyses were done, each resulting from 12 to 20 successive readings (10 seconds photon counts/reading) averaged to obtain a data set. The number of successive readings required for each analysis was optimized by the apparatus. Mean sizes in intensity were derived from correlation functions obtained from in the data set. Compositions containing no lyoprotectant showed aggregation upon freeze-thawing, with particle size increasing about 5-fold. Compositions containing at least 1% (w/v) mannitol, 0.5% (w/v) sucrose, 0.5% (w/v) dextran 5 kDa, or 0.1% (w/v) trehalose dihydrate maintained nanoparticle mean size in intensity below 150 nm upon freeze-thawing. At higher lyoprotectant contents, no variation in size was seen among samples (±125 nm). Mannitol was the least efficient lyoprotectant, with particles greater than 300 nm following freeze-thaw at concentrations of 0.1 or 0.5% (w/v) (FIG. 1A).

4—ESEM Imaging

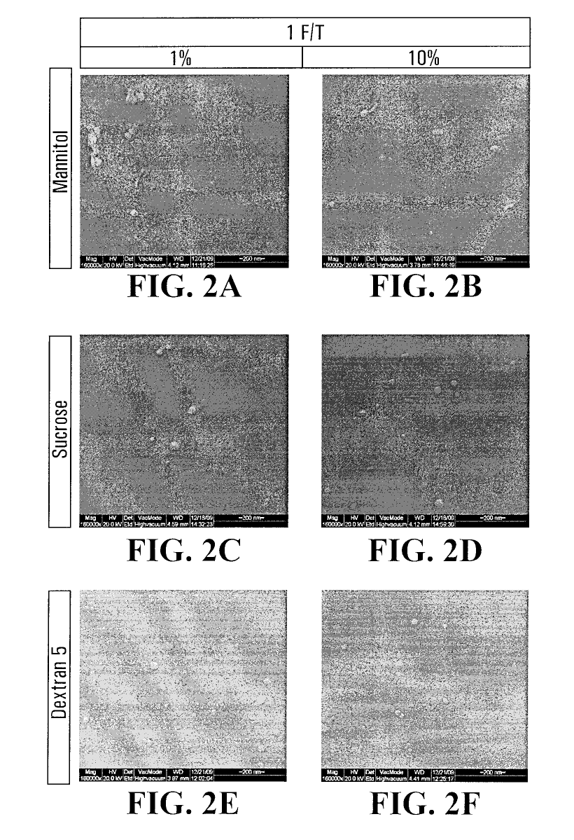

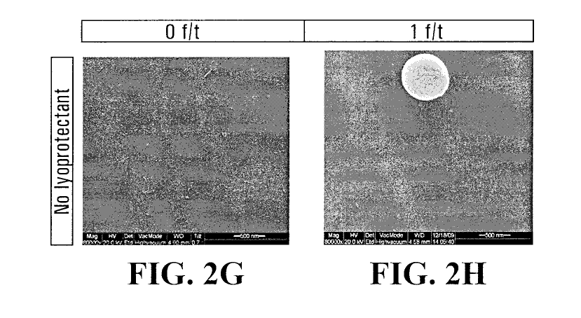

Compositions with no lyoprotectant were observed pre- and post-freeze-thaw, while samples containing low (1% (w/v)) and high (10% (w/v)) mannitol, sucrose or dextran 5 were observed post freeze-thaw. Small volumes of sample were pulverized on polished silicon wafers using a gas spray method, and then were sputter-coated with gold. Observations were performed using the high vacuum mode of the Environmental Scanning Electron Microscope (ESEM) for greater resolution. The high vacuum observation parameters were as follows: accelerating voltage=20 kV; spot size=3; working distance ˜5 mm. Complexes freshly prepared in absence of lyoprotectant were less than 200 nm in size and had different morphologies (spherical, rod-like or toroidal), while they mostly formed large spherical aggregates (greater than 500 nm) after freeze-thaw. Samples freeze-thawed in 1 or 10% lyoprotectant were more spherical and remained smaller than 200 nm (FIG. 2A-H). Complexes prepared in 1% w/V mannitol seemed slightly larger than those prepared in sucrose or dextran 5, as previously seen in DLS.

5—In Vitro Transfection

Compositions containing mannitol were not tested in vitro since they were the least efficient at preserving particle size upon freeze-thawing, as previously seen. Only compositions shown to preserve particle size below 200 nm upon freeze-thawing, and containing at most 3% (w/v) lyoprotectant, were assayed in vitro. These were: 0.5 to 3% (w/v) sucrose; 0.5 to 3% (w/v) dextran 5; and 0.1 to 3% (w/v) trehalose dihydrate. HEK293 cells, grown in DMEM high glucose at pH7.4, supplemented with 10% fetal bovine serum (FBS), and incubated at 37° C. in 5% CO₂, were used for in vitro studies. 60,000 cells were plated per well of a 24-well plate 24 hours prior to transfection in order to reach about 50% confluency for transfection (about 150000 cells per well). Two wells of 24-well plates were transfected with each sample: one for analysis of transfection efficiency in flow cytometry, the other for luciferase expression quantification. In each well, the exact volume of nanoparticle composition was added, along with transfection medium (DMEM high glucose at pH6.5 supplemented with 10% FBS), in order to have a total 500 μL of transfection medium and sample containing 2.5 pg of DNA. Cells were then incubated 24 hours at 37° C., in 5% CO₂. In each well, transfection medium was replaced with 500 μL of growth medium and cells were incubated an additional 24 hours at 37° C., in 5% CO₂, prior to analysis.

6—Transfection Efficiency

Transfection efficiency was measured using flow cytometry. Sample preparation: growth medium was removed from each well containing a sample to be analyzed; cells were washed with 100 μL phosphate buffered saline (PBS) at pH 7.4; they were trypsinized 5 minutes at 37° C. using 100 μL trypsin/EDTA per well; then 100 μL growth medium was added and the whole sample was transferred into a cytometry tube. Flow cytometry measurements: 20000 events were collected per sample, and fluorescence was detected through 510/20 nm bandpass filters with photomultiplier tubes following excitation of enhanced green fluorescence protein (EGFP) in transfected cells using a 488 nm argon laser. HEK293 cell line auto-fluorescence was measured using non transfected cells, and fluorescence detection gates were adjusted accordingly. Forward scatter (FSC) and side scatter (SSC) were also used to exclude dead cells and debris from events recorded. Finally, FSC was used to identify and exclude doublets from the events when analyzing transfection efficiency using the data. The percentage of transfected cells in the freeze-thawed compositions was expressed relative to the percentage of transfected cells obtained with freshly prepared complexes containing no lyoprotectant (0FT), which had a transfection efficiency of 43% of total cells. All compositions containing lyoprotectant tested in vitro preserved transfection efficiency after freeze-thawing, with transfection levels at least 87% of that of freshly prepared complexes. Nanoparticles freeze-thawed in absence of lyoprotectant had transfection levels of only 25% that of freshly prepared complex (FIG. 1B).

7—Luciferase Expression

Luciferase expression, in relative light units per minute (RLU/min), was measured using the Bright-Glo™ Luciferase Assay, and was normalized over the total protein content in each sample, measured with the Bicinchoninic acid (BCA) assay. Sample preparation: growth medium was removed from each well containing a sample to be analyzed; cells were washed twice with 100 μL PBS at pH 7.4; cells were lysed 5 minutes at room temperature using 100 μL Glo Lysis Buffer per well; and then cell lysates were stored at −20° C. until analysis. Lysates were thawed at room temperature before use. Luciferase expression quantification: in a white 96-well plate, 25 μL of Bright-Glo™ Luciferase Reagent was mixed with 25 μL of cell lysate; then luminescence was measured. Protein content quantification: in a clear 96-well plate, 200 μL of BCA working reagent was mixed with 25 μL of cell lysate; samples were incubated 30 minutes at 37° C., 5% CO₂, and then cooled to room temperature; absorbance at 562 nm was measured. A standard curve, prepared using serial dilutions of a 200 μg/mL bovine serum albumin (BSA) standard, was prepared and analyzed alongside samples to convert absorbance readings to protein concentrations. Luciferase expression levels were normalized over the value obtained for fresh chitosan/DNA complexes in absence of lyoprotectants (0FT), which had an expression level of 8.03E10 RLU/min·mg of proteins. Luciferase expression was similar between fresh complexes and complexes freeze-thawed in presence of lyoprotectant, with the exception of samples formulated with 1% and 3% (w/v) trehalose, which, respectively, had expression levels 40 to 60% lower than the fresh control. Samples freeze-thawed in absence of lyoprotectant expressed 75% less luciferase than the fresh control (FIG. 1C).

TABLE 2
Performance of the 22 different compositions tested in example 1.

| Criteria | | |
|---|---|---|
| number | Criteria description | Composition Performance |
| 1 | The nanoparticle average size should be below 250 nm (Assessed by DLS) | Passed: #4-23 |
| 2 | The nanoparticle average PDI should be at most 0.25 (Assessed by DLS) | Not checked |
| 3 | The nanoparticle average zeta potential should be positive and sufficient to | Not checked |
| | ensure composition short-term stability (Assessed by LDV) | |
| 4 | There should be no aggregation in the samples (Assessed by ESEM) | Passed: #4, 7, 10, 13, 16, 19 |
| | | Failed: #1 |
| | | Not checked: others |
| 5 | The composition transfection level should be greater than 50% of the | Passed: #9-11, 15-17, 20-23 |
| | transfection level of fresh CS/DNA particles without excipients (Assessed by | Failed: #1 |
| | flow cytometry) | Not checked: others |
| 6 | The composition luciferase expression level should be greater than 50% of the | Passed: #9-11, 15-17, 20-22 |
| | expression level of fresh CS/DNA particles without excipients | Failed: #1, 23 |
| | (Assessed by luminometry) | Not checked: others |
| 7 | The freeze-dried cake should be completely reconstituted within 5 minutes | NA (compositions not dried) |
| | (Assessed with visual inspection upon reconstitution) | |
| 8 | The final DNA concentration should be at least 0.5 mg/mL | Failed: all (concentrations tested |
| | (Assessed from the initial DNA content and the rehydration factor used) | were 0.025 mg/ml) |
| 9 | The rehydrated compositions should be near iso-osmolality: between 200 and | Not checked |
| | 400 mOsm (Assessed with the osmolality model of the compositions) | |
| 10 | The rehydrated compositions should have a near-neutral pH: between 6 and 7 | Not checked |
| | (Assessed with a pH meter) | |

### Example 2

Low Lyoprotectant Content Compositions that Prevented Nanoparticle Aggregation upon Freeze-Thawing Cannot Prevent Aggregation During Freeze-Drying

1—Preparation of Chitosan/DNA Nanoparticle Compositions

Nanoparticle compositions containing no lyoprotectant (composition #1) or containing 0.5% (w/v) sucrose (composition #9), 0.5% (w/v) dextran 5 (Composition #15), or 0.5% (w/v) trehalose dihydrate (composition #21), were prepared as described in Example 1. Samples to be freeze-dried were transferred to 2 mL serum vials and freeze-dried with 13 mm butyl lyophilization stoppers and a water permeable membrane was placed over the tray containing all samples to prevent dust or bacterial contamination.

2—Sample Freeze-Drying

Freeze-drying was carried in a Millrock Laboratory Series Freeze-Dryer PC/PLC, using the following cycle: ramped freezing from room temperature to −40° C. in 1 hour, then maintaining isothermal at −40° C. for 2 hours; primary drying for 48 hours at −40° C., at 100 millitorrs; and secondary drying at 100 millitorrs, increasing temperature to 30° C. in 12 hours and then maintaining isothermal at 30° C. for 6 hours. Samples were stoppered, crimped and stored at 4° C. until use. 15 to 30 minutes prior to use, samples were rehydrated using a volume of Milli-Q water equal to their initial volume before freeze-drying. Although all samples rehydrated within 5 minutes; rehydration was instantaneous with lyoprotectant and slightly slower without any lyoprotectant.

3—DLS Measurements

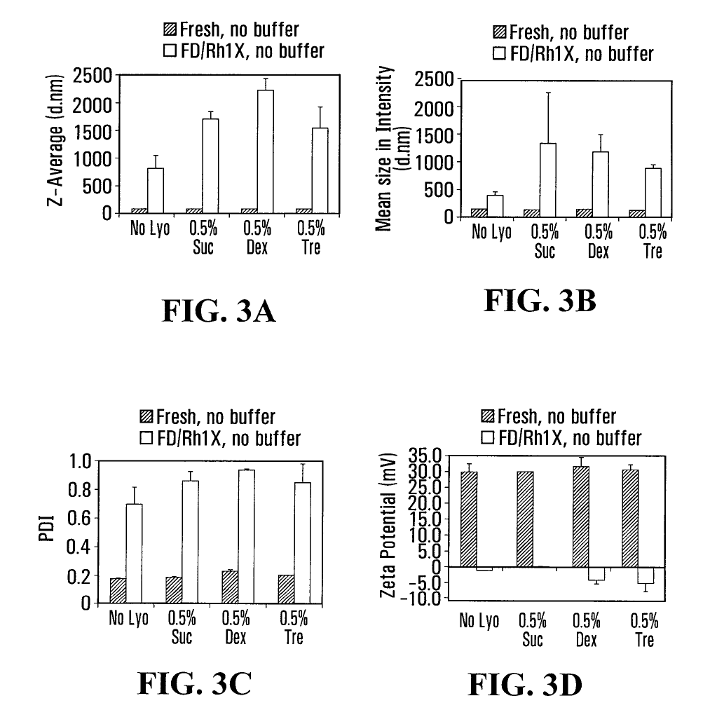

Four samples were analyzed per composition: two freshly prepared and two following a freeze-drying and rehydration to initial volume. For each sample, two or three consecutive size analyses were done, each resulting from 12 to 20 successive readings (10 seconds photon counts/reading) averaged to obtain a data set. The number of successive readings required for each analysis was optimized by the apparatus. Z-average diameter, mean sizes in intensity and PDI were derived from the correlation function obtained from in the data set. All freeze-dried and rehydrated compositions yielded large aggregates as compared to freshly prepared nanoparticles: Z-averages increased by up to 24-fold (FIG. 3A), mean size in intensity increased by up to 9.5-fold (FIG. 3B), and PDI values were above 0.7, an average increase of about 4-fold (FIG. 3C).

4—Zeta Potential Measurements

Four samples were analyzed per composition: two freshly prepared and two following a freeze-drying and rehydration to initial volume. Freeze-dried samples were rehydrated using a volume of Milli-Q water equal to their volume before freeze-drying, and then they were left to stabilize for 15 to 30 minutes. Fresh samples and rehydrated samples were supplemented with 400 μL 20 mM NaCl and, if necessary, their volume was completed to 800 μL with Milli-Q prior to Zeta potential analysis. Zeta potential was measured by Laser Doppler Velocimetry (LDV). For each sample, three consecutive zeta potential analyses were done, each resulting from 10 to 20 successive readings averaged to obtain a data set. The number of successive readings required for each analysis was optimized by the apparatus. All freshly prepared compositions had zeta potentials between 30 and 32 mV, therefore lyoprotectants had no impact on the surface charge of the nanoparticles. Freeze-dried and rehydrated compositions had zeta potentials of 0 to −5 mV (FIG. 3D).

TABLE 3
Performance of the 4 different compositions.

| Criteria | | |
|---|---|---|
| number | Criteria description | Performance |
| 1 | The nanoparticle Z-average should be below 250 nm (Assessed by DLS) | Passed: all fresh |
| | | Failed: all Rh1X |
| 2 | The nanoparticle average PDI should be at most 0.25 (Assessed by DLS) | Passed: all fresh |
| | | Failed: all Rh1X |
| 3 | The nanoparticle average zeta potential should be positive and sufficient to ensure | Passed: all fresh |
| | composition short-term stability (Assessed by LDV) | Failed: all Rh1X |
| 4 | There should be no aggregation in the samples (Assessed by ESEM) | Not checked |
| 5 | The composition transfection level should be greater than 50% of the transfection | Not checked |
| | level of fresh CS/DNA particles without excipients (Assessed by flow cytometry) | |
| 6 | The composition luciferase expression level should be greater than 50% of the | Not checked |
| | expression level of fresh CS/DNA particles without excipients (Assessed by | |
| | luminometry) | |
| 7 | The freeze-dried cake should be completely reconstituted within 5 minutes | Passed: all |
| | (Assessed with visual inspection upon reconstitution) | |
| 8 | The final DNA concentration should be at least 0.5 mg/mL | Failed: all (concentrations |
| | (Assessed from the initial DNA content and the rehydration factor used) | tested were 0.025 mg/ml) |
| 9 | The rehydrated compositions should be near iso-osmolality: between 200 and 400 | Not checked |
| | mOsm (Assessed with the osmolality model of the compositions) | |
| 10 | The rehydrated compositions should have a near-neutral pH: between 6 and 7 | Not checked |
| | (Assessed with a pH meter) | |

### Example 3

The Citric Acid/Trisodium Citrate Buffer System is Not Compatible with Chitosan-Based Compositions—Trisodium Citrate Promotes Chitosan Gelation

1—Preparation of Chitosan/DNA Nanoparticle Compositions

Chitosan (Mn 10 kDa, 92% DDA) was dissolved in HCl overnight at room temperature to obtain a final chitosan concentration of 5 mg/mL. The stock solution was diluted to 271 μg/ml, and then 100 μL was mixed with 100 μL of plasmid DNA (pEGFPLuc) at 100 μg/mL in order to form complexes at N/P ratio of 5. Mixing was performed by pipetting the solution up and down approximately 10 times immediately following addition of chitosan. Samples were left to stabilize at room temperature for 30 minutes, and then sample volumes were completed to 400 μL with sterile 2% (w/v) sucrose, 2% (w/v) dextran 5 kDa, or 2% (w/v) trehalose dihydrate, and sterile 70 mM citric acid/trisodium citrate buffer at pH 4.5 or 6.5, as per Table 4.

TABLE 4
Compositions containing lyoprotectants and citric acid/trisodium citrate buffer.

| | | | Volume added in each sample (μL) | | | | | | |
|---|---|---|---|---|---|---|---|---|---|
| | | | | | | | | 70 mM Citric acid/ | |
| Composition | | | Chitosan | DNA | Suc | Dex | Tre | trisodium citrate | |
| # | pH | Lyo. | 271 μg/mL | 100 μg/mL | 2% (w/v) | 2% (w/v) | 2% (w/v) | pH4.5 | pH6.5 |
| 1 | 4.5 | 0.5% suc | 100 | 100 | 100 | 0 | 0 | 100 | 0 |
| 2 | | 0.5% dex | 100 | 100 | 0 | 100 | 0 | 100 | 0 |
| 3 | | 0.5% tre | 100 | 100 | 0 | 0 | 100 | 100 | 0 |
| 4 | 6.5 | 0.5% suc | 100 | 100 | 100 | 0 | 0 | 0 | 100 |
| 5 | | 0.5% dex | 100 | 100 | 0 | 100 | 0 | 0 | 100 |
| 6 | | 0.5% tre | 100 | 100 | 0 | 0 | 100 | 0 | 100 |

2—Sample Freeze-Thawing

Sample were freeze-thawed as described in Example 1.

3—DLS Measurements

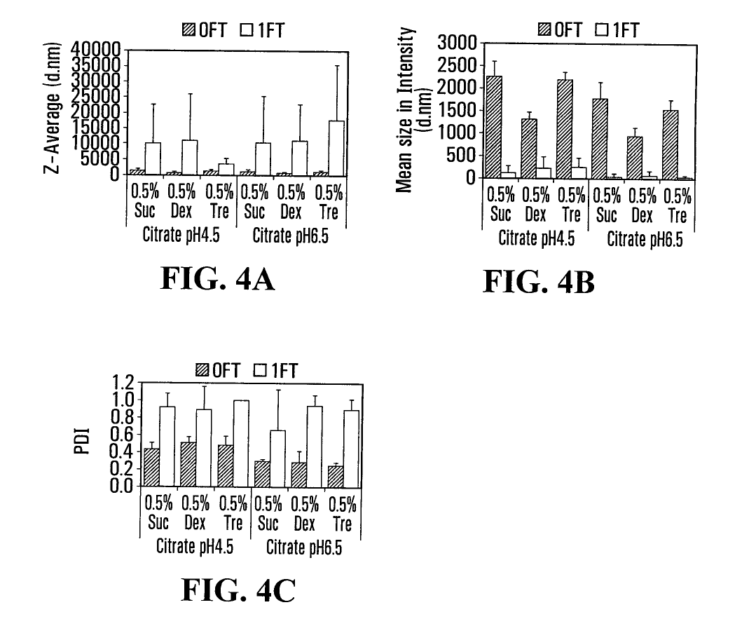

Fresh and freeze-thawed samples prepared were analyzed for size and PDI as described in Example 2. Compositions containing citric acid/trisodium citrate formed large particles prior to freeze-thawing, with mean sizes in intensity larger than 900 nm (FIG. 4B). After freeze-thawing, samples were totally aggregated and unsuitable for DLS analysis, with Z-averages above 3500 nm (FIG. 4A) and PDI values above 0.66 (FIG. 4C).

4—Citric Acid/Trisodium Citrate Incompatibility

Chitosan, sucrose or trehalose dihydrate were mixed with citric acid/trisodium citrate buffer at pH 6.2, or chitosan was mixed with citric acid or trisodium citrate only, as per Table 5.

TABLE 5
Samples prepared to evaluate citric acid/trisodium citrate incompatibility with the compositions.

| ID | Composition | Final concentration of each component | |
|---|---|---|---|
| S1 | Chitosan + Citric acid/trisodium citrate | Chitosan: 1 mg/mL | Citric acid/trisodium citrate: 35 mM |
| S2 | Chitosan + Citric acid | Chitosan: 1 mg/mL | Citric acid: 2 mM |
| S3 | Chitosan + Trisodium citrate | Chitosan: 1 mg/mL | Trisodium citrate: 33 mM |
| S4 | Sucrose + Citric acid/trisodium citrate | Sucrose: 3% (w/v) | Citric acid/trisodium citrate: 35 mM |
| S5 | Trehalose + Citric acid/trisodium citrate | Trehalose: 3% (w/v) | Citric acid/trisodium citrate: 35 mM |

Chitosan solutions became turbid in presence of buffer or trisodium citrate, but not in presence of citric acid (data not shown). Turbidity was maximal in presence of trisodium citrate, with white cloud-like structures forming in the solution upon gelation of the chitosan/trisodium citrate mixture (data not shown). Gelation may be caused by cross-linking of positively charged chitosan chains by negatively charged trivalent trisodium citrate (data not shown).

TABLE 6
Performance of the 6 different compositions.

| Criteria | | |
|---|---|---|
| number | Criteria description | Performance |
| 1 | The nanoparticle Z-average should be below 250 nm (Assessed by DLS) | Failed: all |
| 2 | The nanoparticle average PDI should be at most 0.25 (Assessed by DLS) | Failed: all |
| 3 | The nanoparticle average zeta potential should be positive and sufficient to ensure | Not checked |
| | composition short-term stability (Assessed by LDV) | |
| 4 | There should be no aggregation in the samples (Assessed by ESEM) | Not checked |
| 5 | The composition transfection level should be greater than 50% of the transfection level of | Not checked |
| | fresh CS/DNA particles without excipients (Assessed by flow cytometry) | |
| 6 | The composition luciferase expression level should be greater than 50% of the expression | Not checked |
| | level of fresh CS/DNA particles without excipients (Assessed by luminometry) | |
| 7 | The freeze-dried cake should be completely reconstituted within 5 minutes | NA (compositions not |
| | (Assessed with visual inspection upon reconstitution) | dried) |
| 8 | The final DNA concentration should be at least 0.5 mg/mL | Failed: all |
| | (Assessed from the initial DNA content and the rehydration factor used) | (concentrations tested |
| | | were 0.025 mg/ml) |
| 9 | The rehydrated compositions should be near iso-osmolality: between 200 and 400 mOsm | Not checked |
| | (Assessed with the osmolality model of the compositions) | |
| 10 | The rehydrated compositions should have a near-neutral pH: between 6 and 7 | Not checked |
| | (Assessed with a pH meter) | |

### Example 4

L-Histidine is Compatible with Chitosan-Based Compositions and Leads to Nanoparticle Suspensions with Lower Polydispersity Indexes

1—Preparation of Chitosan/DNA Nanoparticle Compositions for ESEM Imaging

Chistosan/DNA complexes were prepared as described in Example 3. Following complex stabilization at room temperature for 30 minutes, sample volumes were completed to 400 μL with sterile 4% (w/v) sucrose, 4% (w/v) dextran 5 kDa, or 4% (w/v) trehalose dihydrate, sterile 55 mM L-histidine buffer at pH 6.5 or Milli-Q water, as per Table 7.

TABLE 7
Compositions with lyoprotectants and L-histidine for ESEM imaging.

| | | | Volume added in each sample (μL) | | | | | | |
|---|---|---|---|---|---|---|---|---|---|
| | | | | | Suc | Dex | Tre | L-histidine | |
| Composition | | | Chitosan | DNA | 4% | 4% | 4% | pH6.5, | Milli-Q |
| # | Buffer | Lyo. | 271 μg/mL | 100 μg/mL | (w/v) | (w/v) | (w/v) | 55 mM | water |
| 1 | None | None | 100 | 100 | 0 | 0 | 0 | 0 | 200 |
| 2 | 13.75 mM | None | 100 | 100 | 0 | 0 | 0 | 100 | 100 |
| | His | | | | | | | | |
| 3 | None | 1% suc | 100 | 100 | 100 | 0 | 0 | 0 | 100 |
| 4 | | 1% dex | 100 | 100 | 0 | 100 | 0 | 0 | 100 |
| 5 | | 1% tre | 100 | 100 | 0 | 0 | 100 | 0 | 100 |
| 6 | 13.75 mM | 1% suc | 100 | 100 | 100 | 0 | 0 | 100 | 0 |
| 7 | His | 1% dex | 100 | 100 | 0 | 100 | 0 | 100 | 0 |
| 8 | | 1% tre | 100 | 100 | 0 | 0 | 100 | 100 | 0 |

2—ESEM Imaging

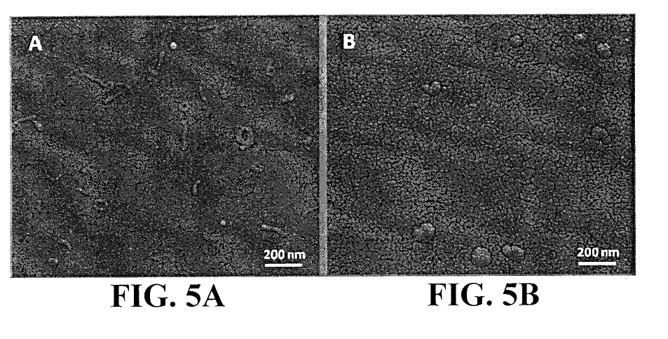

ESEM sample preparation and imaging was performed as described in Example 1. Fresh nanoparticles formulated in absence of lyoprotectant or histidine had spherical, rod-like or toroidal morphologies (FIG. 5A), whereas they were more spherical following addition of L-histidine at pH6.5 and final concentration of 13.75 mM (FIG. 5B). Formulating complexes in 1% (w/v) lyoprotectant, with or without 13.75 mM histidine, had a similar impact on the nanoparticles observed.

3—Preparation of Chitosan/DNA Nanoparticle Compositions for DLS Analysis After Freeze-Thawing

Chitosan/DNA complexes were prepared as described in Example 3. Following complex stabilization at room temperature for 30 minutes, sample volumes were completed to 400 μL with sterile 2% (w/v) sucrose, 2% (w/v) dextran 5 kDa, or 2% (w/v) trehalose dihydrate, and sterile 55 mM L-histidine buffer at pH 6.5 or Milli-Q water, as per Table 8. Samples were freeze-thawed as described in Example 1.

TABLE 8
Compositions with lyoprotectants and L-histidine for the freeze-thawing study.

| | | | Volume added in each sample (μL) | | | | | | |
|---|---|---|---|---|---|---|---|---|---|
| | | | Chitosan | DNA | Suc | Dex | Tre | L-histidine | Milli- |
| Composition | | | 271 | 100 | 2% | 2% | 2% | pH6.5, | Q |
| # | Buffer | Lyo. | μg/mL | μg/mL | (w/v) | (w/v) | (w/v) | 55 mM | water |
| 9 | None | 0.5% suc | 100 | 100 | 100 | 0 | 0 | 0 | 100 |
| 10 | | 0.5% dex | 100 | 100 | 0 | 100 | 0 | 0 | 100 |
| 11 | | 0.5% tre | 100 | 100 | 0 | 0 | 100 | 0 | 100 |
| 12 | 13.75 mM | 0.5% suc | 100 | 100 | 100 | 0 | 0 | 100 | 0 |
| 13 | His | 0.5% dex | 100 | 100 | 0 | 100 | 0 | 100 | 0 |
| 14 | | 0.5% tre | 100 | 100 | 0 | 0 | 100 | 100 | 0 |

4—DLS Measurements

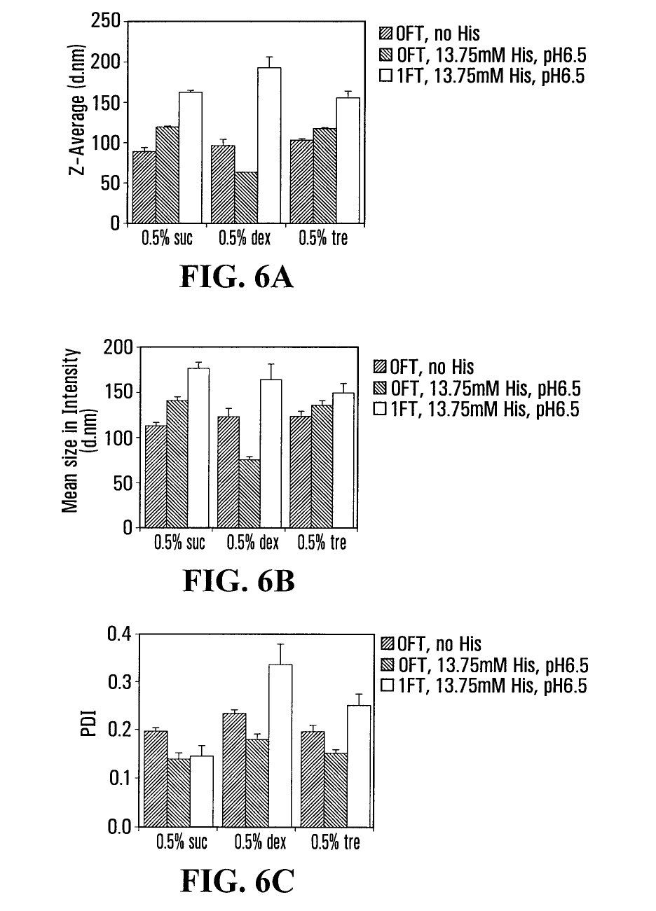

Duplicates of each composition without histidine (compositions #9 to 11) were analyzed freshly prepared; duplicates of each composition with histidine (compositions #12 to 14) were analyzed freshly prepared and after freeze-thawing. Size and PDI analyses were done as described in Example 2. Addition of histidine had little impact on fresh compositions (FIGS. 6A-C): Z-average increased by 30 and 13 nm in presence of sucrose and trehalose respectively, and decreased by 34 nm in presence of dextran; mean size in intensity increased by 12 and 29 nm in presence of sucrose and trehalose respectively, and decreased by 48 nm in presence of dextran; and PDI decreased by 0.05 in presence of all lyoprotectants. No adverse reaction was seen in compositions upon freeze-thawing in presence of histidine (FIGS. 6A-C): Z-averages and mean sizes in intensity were below 200 nm and average PDI values were below 0.35.

TABLE 9
Performance of the 14 different compositions.

| Criteria | | |
|---|---|---|
| number | Criteria description | Performance |
| 1 | The nanoparticle Z-average should be below 250 nm (Assessed by DLS) | Passed: #9-14 |
| | | Not checked: #1-8 |
| 2 | The nanoparticle average PDI should be at most 0.25 (Assessed by DLS) | Passed: #9-12, 14 |
| | | Failed: #13 |
| | | Not checked: #1-8 |
| 3 | The nanoparticle average zeta potential should be positive and sufficient to | Not checked |
| | ensure composition short-term stability (Assessed by LDV) | |
| 4 | There should be no aggregation in the samples (Assessed by ESEM) | Passed: #1-8 |
| | | Not checked: #9-14 |
| 5 | The composition transfection level should be greater than 50% of the | Not checked |
| | transfection level of fresh CS/DNA particles without excipients | |
| | (Assessed by flow cytometry) | |
| 6 | The composition luciferase expression level should be greater than 50% of the | Not checked |
| | expression level of fresh CS/DNA particles without excipients | |
| | (Assessed by luminometry) | |
| 7 | The freeze-dried cake should be completely reconstituted within 5 minutes | NA (compositions not |
| | (Assessed with visual inspection upon reconstitution) | dried) |
| 8 | The final DNA concentration should be at least 0.5 mg/mL | Failed: all (concentrations |
| | (Assessed from the initial DNA content and the rehydration factor used) | tested were 0.025 mg/ml) |
| 9 | The rehydrated compositions should be near iso-osmolality: between 200 and | Not checked |
| | 400 mOsm (Assessed with the osmolality model of the compositions) | |
| 10 | The rehydrated compositions should have a near-neutral pH: between 6 and 7 | Not checked |
| | (Assessed with a pH meter) | |

### Example 5

L-Histidine Prevents Nanoparticle Aggregation Following Freeze-Drying when Added to Compositions Containing Lyoprotectant

Compositions can be concentrated up to 20-fold without significant changes to nanoparticle size and PDI; and

L-histidine can be minimized in compositions while still preventing particle aggregation following freeze-drying.

1—Preparation of Chitosan/DNA Nanoparticle Compositions for Freeze-Drying and Rehydration to Higher Concentrations

Chitosan/DNA complexes were prepared as described in Example 3. Following complex stabilization at room temperature for 30 minutes, sample volumes were completed to 400 μL with sterile 2% (w/v) sucrose, 2 or 4% (w/v) dextran 5 kDa or trehalose dihydrate, and 55 mM L-histidine buffer at pH 6.5, as per Table 10.

TABLE 10
Compositions to be freeze-dried and rehydrated at higher concentrations.

| | | Volume added in each sample (μL) | | | | | | | |
|---|---|---|---|---|---|---|---|---|---|
| | | Chitosan | DNA | | | | | | L-histidine |
| Composition | | 271 | 100 | Sucrose | Dextran | | Trehalose | | pH6.5, |
| # | Description | μg/mL | μg/mL | 2% | 2% | 4% | 2% | 4% | 55 mM |
| 1 | 0.5% suc-his(13.75) | 100 | 100 | 100 | 0 | 0 | 0 | 0 | 100 |
| 2 | 0.5% dex-his(13.75) | 100 | 100 | 0 | 100 | 0 | 0 | 0 | 100 |
| 3 | 1% dex-his(13.75) | 100 | 100 | 0 | 0 | 100 | 0 | 0 | 100 |
| 4 | 0.5% tre-his(13.75) | 100 | 100 | 0 | 0 | 0 | 100 | 0 | 100 |
| 5 | 1% tre-his(13.75) | 100 | 100 | 0 | 0 | 0 | 0 | 100 | 100 |

For Rh1×, Rh5× and Rh10×: six samples of composition #3 to 5 (see Table 10) were prepared and freeze-dried as described in Example 2; for each composition, two samples were rehydrated to their original volume with 400 μL Milli-Q (Rh1×), two were rehydrated 5 times more concentrated with 80 μL Milli-Q (Rh5×), and two were rehydrated 10 times more concentrated with 40 μL Milli-Q (Rh10×). For Rh1× and Rh20×: four samples of composition #1, 2 and 4 (see Table 10) were prepared and freeze-dried as described in Example 2; for each composition, two samples were rehydrated to their original volume with 400 μL Milli-Q (Rh1×) and two were rehydrated 20 times more concentrated with 20 μL Milli-Q (Rh20×). Rehydrated samples were left to stabilize 15 to 30 minutes prior to analysis. All samples rehydrated within 5 minutes, although Rh20× was harder to achieve given the small rehydration volume relative to the cake volume.

2—DLS Measurements of Compositions Rehydrated to Higher Concentrations

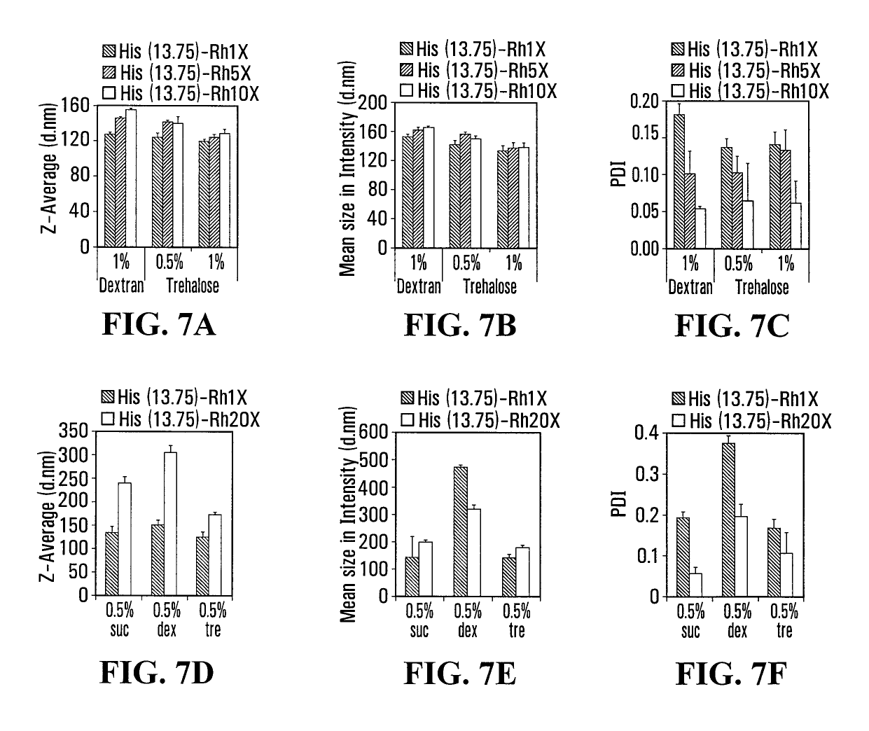

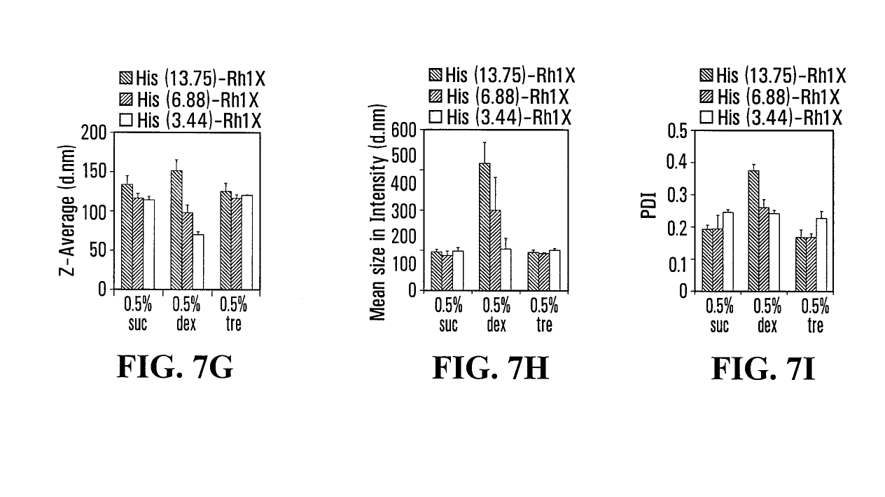

Size and PDI analyses were done as described in Example 2. Rehydration of compositions #3 to 5 (13.75 mM histidine with 1% (w/v) dextran, or 0.5 or 1% (w/v) trehalose dihydrate) up to 10 times more concentrated (Rh1× to Rh10×) had no impact on particle Z-average, which varied from 120 to 155 nm (FIG. 7A), or mean size in intensity, which varied from 131 to 165 nm (FIG. 7B). Nanoparticle PDI values decreased from 0.18 to 0.05 upon increasing the concentration factor (FIG. 7C). Compositions containing 0.5% sucrose or trehalose, combined to 13.75 mM histidine, rehydrated at 20 times their initial concentration yielded particles smaller than 250 nm (Z-average, FIG. 7D) or 200 nm (mean size in intensity, FIG. 7E); compositions containing dextran and Rh20× had a Z-average of 305 nm (FIG. 7D) and a mean size in intensity of 324 nm (FIG. 7E). All compositions had PDI values below 0.2, except 0.5% dextran Rh1× with a PDI of 0.37; nanoparticles in compositions Rh20× had PDI values inferior to those in composition RH1× (FIG. 7F).

3—Preparation of Chitosan/DNA Nanoparticle Compositions for Freeze-Drying with Lower Histidine Content

Chitosan/DNA complexes were prepared as described in Example 3. Following complex stabilization at room temperature for 30 minutes, sample volumes were completed to 400 μL with sterile 2% (w/v) sucrose, dextran 5 kDa or trehalose dihydrate, and 55, 27.5 or 13.75 mM L-histidine buffer at pH 6.5, as per Table 11.

TABLE 11
Compositions to be freeze-dried and rehydrated at higher concentrations.

| | | | Volume added in each sample (μL) | | | | | | | |
|---|---|---|---|---|---|---|---|---|---|---|
| Composition | | | Chitosan | DNA | Suc | Dex | Tre | L-histidine pH6.5 | | |
| # | Lyo | Buffer | 271 μg/mL | 100 μg/mL | 2% | 2% | 2% | 55 mM | 27.5 mM | 13.75 mM |
| 6 | 0.5% | His(13.75) | 100 | 100 | 100 | 0 | 0 | 100 | 0 | 0 |
| 7 | suc | His(6.88) | 100 | 100 | 100 | 0 | 0 | 0 | 100 | 0 |
| 8 | | His(3.44) | 100 | 100 | 100 | 0 | 0 | 0 | 0 | 100 |
| 9 | 0.5% | His(13.75) | 100 | 100 | 0 | 100 | 0 | 100 | 0 | 0 |
| 10 | dex | His(6.88) | 100 | 100 | 0 | 100 | 0 | 0 | 100 | 0 |
| 11 | | His(3.44) | 100 | 100 | 0 | 100 | 0 | 0 | 0 | 100 |
| 12 | 0.5% | His(13.75) | 100 | 100 | 0 | 0 | 100 | 100 | 0 | 0 |
| 13 | tre | His(6.88) | 100 | 100 | 0 | 100 | 0 | 100 | 0 | |
| 14 | | His(3.44) | 100 | 100 | 0 | 0 | 100 | 0 | 0 | 100 |

Duplicates of each composition were prepared and freeze-dried as described in Example 2, and then were rehydrated to their original volume with 400 μL Milli-Q (Rh 1×).

4—DLS Measurements of Compositions Freeze-Dried with Lower Histidine Content

Size and PDI analyses were done as described in Example 2. Decreasing the histidine content had no impact on particle size of rehydrated compositions containing 0.5% (w/v) sucrose or trehalose dihydrate, with particles of less than 160 nm in diameter (FIGS. 7G-H). The PDI of these two compositions remained inferior or equal to 0.25 when reducing the histidine content from 13.75 to 3.44 mM (FIG. 7I). Z-average and mean size in intensity of dextran compositions Rh1× decreased from 151 to 71 nm and from 477 to 157 nm respectively upon reduction of histidine concentration from 13.75 to 3.44 mM (FIGS. 7G-H). The average PDI of these compositions decreased from 0.37 to 0.25 upon reduction of histidine content from 13.75 to 6.88 mM, with final PDI of 0.24 at 3.44 mM histidine (FIG. 7I).

5—Osmolality Model and Estimates

A model was developed to estimate composition osmolalities, given the large volume of fresh samples required to measure osmolalities of freeze-dried samples rehydrated in 20 times less volume (20-fold concentration). Assuming nanoparticle osmolality is negligible, the model was established using serial dilutions of excipients only (sucrose, dextran 5 kDa, trehalose dihydrate and L-histidine). The resulting model predicted the osmolality of a composition containing 5% (w/v) dextran, 5% (w/v) trehalose dihydrate, and 35 mM L-histidine at pH 6.5, with a precision of 1.8%. Based on the model, osmolalities varied between 4 and 570 mOsm, depending on the lyoprotectant, the histidine content, and the concentration factor upon rehydration. Osmolalities were higher for compositions containing sucrose and lower for those containing dextran 5 kDa. Two compositions were close to isotonicity: 0.5% dex−his(13.75)−Rh20× at 279 mOsm, and 0.5% dex−his(13.75)−Rh10× at 268 mOsm.

TABLE 12
Performance of the 14 different compositions.

| Criteria | | |
|---|---|---|
| number | Criteria description | Performance |
| 1 | The nanoparticle Z-average should be below 250 nm (Assessed by DLS) | Passed: #1, 3-14 |
| | | Failed: #2 (Rh20X) |
| 2 | The nanoparticle average PDI should be at most 0.25 (Assessed by DLS) | Passed: #1, 2 (Rh20X), 3-4, |
| | | 5-8, 10-14 |
| | | Failed: #2(Rh1X), 9 |
| 3 | The nanoparticle average zeta potential should be positive and sufficient to | Not checked |
| | ensure composition short-term stability (Assessed by LDV) | |
| 4 | There should be no aggregation in the samples (Assessed by ESEM) | Not checked |
| 5 | The composition transfection level should be greater than 50% of the | Not checked |
| | transfection level of fresh CS/DNA particles without excipients | |
| | (Assessed by flow cytometry) | |
| 6 | The composition luciferase expression level should be greater than 50% of the | Not checked |
| | expression level of fresh CS/DNA particles without excipients | |
| | (Assessed by luminometry) | |
| 7 | The freeze-dried cake should be completely reconstituted within 5 minutes | Passed: all |
| | (Assessed with visual inspection upon reconstitution) | |
| 8 | The final DNA concentration should be at least 0.5 mg/mL | Passed: 1, 2, 4 (concentrations |
| | (Assessed from the initial DNA content and the rehydration factor used) | at 0.5 mg/ml after Rh20X) |
| | | Failed: others (concentrations |
| | | at 0.025 mg/ml fresh or after |
| | | Rh1X; at 0.125 after Rh5X; |
| | | and at 0.25 after Rh10X) |
| 9 | The rehydrated compositions should be near iso-osmolality: between 200 and | Passed: #2(Rh20X), 4(Rh10X) |
| | 400 mOsm (Assessed with the osmolality model of the compositions) | Failed: Others |
| 10 | The rehydrated compositions should have a near-neutral pH: between 6 and 7 | Not checked |
| | (Assessed with a pH meter) | |

### Example 6

Nanoparticle concentration in fresh composition can be maximized by adding lyoprotectants and buffer to nucleic acid and chitosan prior to complex formation; Minimizing lyoprotectant and buffer contents in these compositions allow reconstitution of cakes to higher concentration while remaining near-isotonic; and

These compositions can be concentrated up to 20 fold without significant changes to nanoparticle physico-chemical properties and transfection efficiency;

1—Preparation of Concentrated Chitosan/DNA Nanoparticle Compositions Containing 13.75 mM Histidine

Chitosan (Mn 10 kDa, 92% DDA) was dissolved in HCl overnight at room temperature to obtain a final chitosan concentration of 5 mg/mL. The chitosan stock solution was diluted to 271 μg/ml using sterile lyoprotectant solutions (2 or 4% (w/v) sucrose, dextran 5 kDa, or trehalose dihydrate), sterile 55 mM L-histidine buffer at pH 6.5 and Milli-Q water, as per Table 13.

TABLE 13
Chitosan dilution with excipients prior to complex formation.

| | | Volume added in each sample (μL) | | | | | | | | |
|---|---|---|---|---|---|---|---|---|---|---|
| | | Chitosan | | | | | | | L-histidine | Milli- |
| Composition | | 5 mg/ | Sucrose | | Dextran | | Trehalose | | pH6.5, | Q |
| # | Description | mL | 2% | 4% | 2% | 4% | 2% | 4% | 55 mM | water |
| 1 | No Lyo | 20 | 0 | 0 | 0 | 0 | 0 | 0 | 0 | 330 |
| 2 | No Lyo-his(13.75) | 20 | 0 | 0 | 0 | 0 | 0 | 0 | 87.5 | 242.5 |
| 3 | 0.5% suc | 20 | 87.5 | 0 | 0 | 0 | 0 | 0 | 0 | 242.5 |
| 4 | 0.5% suc-his(13.75) | 20 | 87.5 | 0 | 0 | 0 | 0 | 0 | 87.5 | 155 |
| 5 | 1% suc | 20 | 0 | 87.5 | 0 | 0 | 0 | 0 | 0 | 242.5 |
| 6 | 1% suc-his(13.75) | 20 | 0 | 87.5 | 0 | 0 | 0 | 0 | 87.5 | 155 |
| 7 | 0.5% dex | 20 | 0 | 0 | 87.5 | 0 | 0 | 0 | 0 | 242.5 |
| 8 | 0.5% dex-his(13.75) | 20 | 0 | 0 | 87.5 | 0 | 0 | 0 | 87.5 | 155 |
| 9 | 1% dex | 20 | 0 | 0 | 0 | 87.5 | 0 | 0 | 0 | 242.5 |
| 10 | 1% dex-his(13.75) | 20 | 0 | 0 | 0 | 87.5 | 0 | 0 | 87.5 | 155 |
| 11 | 0.5% tre | 20 | 0 | 0 | 0 | 0 | 87.5 | 0 | 0 | 242.5 |
| 12 | 0.5% tre-his(13.75) | 20 | 0 | 0 | 0 | 0 | 87.5 | 0 | 87.5 | 155 |
| 13 | 1% tre) | 20 | 0 | 0 | 0 | 0 | 0 | 87.5 | 0 | 242.5 |
| 14 | 1% tre-his(13.75) | 20 | 0 | 0 | 0 | 0 | 0 | 87.5 | 87.5 | 155 |

DNA (pEGFPLuc at 200 μg/mL) stock solution was diluted to 100 μg/ml using sterile lyoprotectant solutions (2 or 4% (w/v) sucrose, dextran 5 kDa, or trehalose dihydrate), 55 mM L-histidine buffer at pH 6.5 and/or Milli-Q water, as per Table 14.

TABLE 14
DNA dilution with excipients prior to complex formation.

| | | Volume added in each sample (μL) | | | | | | | | |
|---|---|---|---|---|---|---|---|---|---|---|
| | | DNA | | | | | | | L-histidine | Milli- |
| Composition | | 200 | Sucrose | | Dextran | | Trehalose | | pH6.5, | Q |
| # | Description | μg/mL | 2% | 4% | 2% | 4% | 2% | 4% | 55 mM | water |
| 1 | No Lyo | 175 | 0 | 0 | 0 | 0 | 0 | 0 | 0 | 175 |
| 2 | No Lyo-his(13.75) | 175 | 0 | 0 | 0 | 0 | 0 | 0 | 87.5 | 87.5 |
| 3 | 0.5% suc | 175 | 87.5 | 0 | 0 | 0 | 0 | 0 | 0 | 87.5 |
| 4 | 0.5% suc-his(13.75) | 175 | 87.5 | 0 | 0 | 0 | 0 | 0 | 87.5 | 0 |
| 5 | 1% suc | 175 | 0 | 87.5 | 0 | 0 | 0 | 0 | 0 | 87.5 |
| 6 | 1% suc-his(13.75) | 175 | 0 | 87.5 | 0 | 0 | 0 | 0 | 87.5 | 0 |
| 7 | 0.5% dex | 175 | 0 | 0 | 87.5 | 0 | 0 | 0 | 0 | 87.5 |
| 8 | 0.5% dex-his(13.75) | 175 | 0 | 0 | 87.5 | 0 | 0 | 0 | 87.5 | 0 |
| 9 | 1% dex | 175 | 0 | 0 | 0 | 87.5 | 0 | 0 | 0 | 87.5 |
| 10 | 1% dex-his(13.75) | 175 | 0 | 0 | 0 | 87.5 | 0 | 0 | 87.5 | 0 |
| 11 | 0.5% tre | 175 | 0 | 0 | 0 | 0 | 87.5 | 0 | 0 | 87.5 |
| 12 | 0.5% tre-his(13.75) | 175 | 0 | 0 | 0 | 0 | 87.5 | 0 | 87.5 | 0 |
| 13 | 1% tre) | 175 | 0 | 0 | 0 | 0 | 0 | 87.5 | 0 | 87.5 |
| 14 | 1% tre-his(13.75) | 175 | 0 | 0 | 0 | 0 | 0 | 87.5 | 87.5 | 0 |

Duplicates of each composition were prepared. For each duplicate, 100 μL of chitosan solution was mixed with 100 μL of its complementary DNA solution (for example, chitosan Composition #1 with DNA Composition #1), in order to form complexes at N/P ratio of 5. Mixing was performed by pipetting the solution up and down approximately 10 times immediately following addition of chitosan. Samples were left to stabilize at room temperature for 30 minutes prior to analysis.

2—DLS Measurements of Concentrated Compositions Containing 13.75 mM Histidine

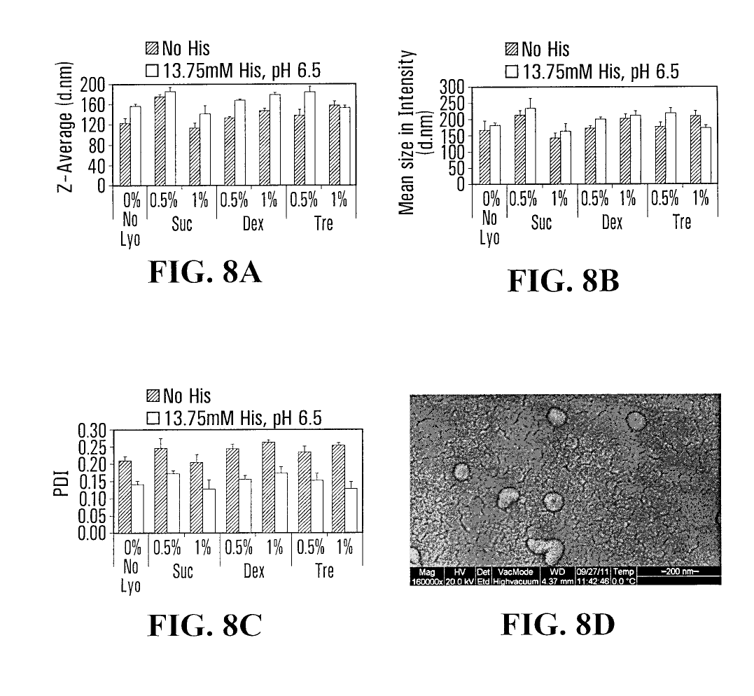

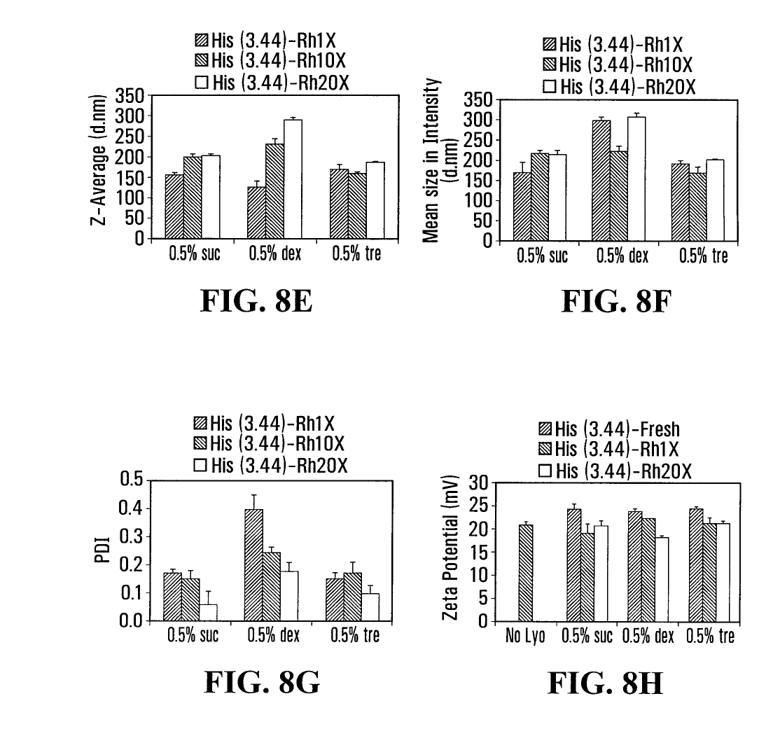

Size and PDI analyses were done as described in Example 2. Addition of lyoprotectant and L-histidine to chitosan or DNA prior to complex formation, rather than after, allowed production of compositions twice less diluted, with Z-averages below 200 nm, mean sizes in intensity below 250 nm, and PDI values below 0.3 (FIGS. 8A-D). Compositions prepared without L-histidine had particle with Z-averages varying between 115 and 176 nm, with mean sizes in intensity between 144 and 214 nm, and with PDI values between 0.21 and 0.26; compositions prepared with L-histidine were slightly larger in sizes, with Z-averages between 143 and 187 nm and mean sizes in intensity between 165 and 237 nm, but had smaller PDI values (0.13 to 0.18) (FIGS. 8A-C). Although addition of excipients to chitosan and DNA prior to complex formation yielded slightly larger particles than previously seen when adding them post nanoparticle formation, PDI values remained similar (see “0FT, no His” and “0FT, 13.75 mM His, pH6.5” in FIGS. 8C-E).

3—Preparation of Concentrated Chitosan/DNA Nanoparticle Compositions Containing 3.44 mM Histidine for Rehydration to Higher Concentrations

Chitosan/DNA complexes were prepared as described in Section 1, but using a histidine stock solution at 13.75 mM instead of 55 mM to dilute chitosan and DNA, as per Tables 15 and 16.

TABLE 15
Chitosan dilution with excipients prior to complex formation in 3.44 mM histidine.

| | | Volume added in each sample (μL) | | | | | |
|---|---|---|---|---|---|---|---|
| | | | | | | L-histidine | |
| Composition | | Chitosan | Suc | Dex | Tre | pH6.5, | Milli-Q |
| # | Description | 5 mg/mL | 2% (w/v) | 2% (w/v) | 2% (w/v) | 13.75 mM | water |
| 15 | No Lyo | 40 | 0 | 0 | 0 | 0 | 660 |
| 16 | No Lyo-his(3.44) | 40 | 0 | 0 | 0 | 175 | 485 |
| 17 | 0.5% suc-his(3.44) | 40 | 175 | 0 | 0 | 175 | 310 |
| 18 | 0.5% dex-his(3.44) | 40 | 0 | 175 | 0 | 175 | 310 |
| 19 | 0.5% tre-his(3.44) | 40 | 0 | 0 | 175 | 175 | 310 |
| 20 | 0.5% suc-his(0) | 40 | 175 | 0 | 0 | 0 | 485 |
| 21 | 0.5% dex-his(0) | 40 | 0 | 175 | 0 | 0 | 485 |
| 22 | 0.5% tre-his(0) | 40 | 0 | 0 | 175 | 0 | 485 |

TABLE 16
DNA dilution with excipients prior to complex formation in 3.44 mM histidine.

| | | Volume added in each sample (μL) | | | | | |
|---|---|---|---|---|---|---|---|
| | | | | | | L-histidine | |
| Composition | | DNA | Suc | Dex | Tre | pH6.5, | Milli-Q |
| # | Description | 200 μg/mL | 2% (w/v) | 2% (w/v) | 2% (w/v) | 13.75 mM | water |
| 15 | No Lyo | 350 | 0 | 0 | 0 | 0 | 350 |
| 16 | No Lyo-his(3.44) | 350 | 0 | 0 | 0 | 175 | 175 |
| 17 | 0.5% suc-his(3.44) | 350 | 175 | 0 | 0 | 175 | 0 |
| 18 | 0.5% dex-his(3.44) | 350 | 0 | 175 | 0 | 175 | 0 |
| 19 | 0.5% tre-his(3.44) | 350 | 0 | 0 | 175 | 175 | 0 |
| 20 | 0.5% suc-his(0) | 350 | 175 | 0 | 0 | 0 | 175 |
| 21 | 0.5% dex-his(0) | 350 | 0 | 175 | 0 | 0 | 175 |
| 22 | 0.5% tre-his(0) | 350 | 0 | 0 | 175 | 0 | 175 |

Samples were freeze-dried as described in Example 2. Rh1× samples were rehydrated with 200 μL of Milli-Q water, Rh10× samples were rehydrated with 20 μL of Milli-Q water, and Rh20× samples were rehydrated with 10 μL of Milli-Q water. All samples rehydrated within 5 minutes, although Rh20× was harder to achieve given the small rehydration volume relative to the cake volume.

4—DLS Measurements of Concentrated Compositions Containing 3.44 mM Histidine, Rehydrated to Higher Concentrations

Six replicates of each of the other compositions (#17 to 19) were prepared as described in Section 3: two Rh1×, two Rh10×, and two Rh20×. Size and PDI analyses were done as described in Example 2. Nanoparticles freeze-dried with only 3.44 mM histidine could be rehydrated up to 20-fold without seeing particle aggregation; particle Z-averages increased by 3 to 68 nm and mean sizes in intensity increased by 7 to 46 nm, as compared to Rh1×, depending on lyoprotectants (FIGS. 8D-E). PDI values decreased when increasing the rehydration concentration factors from 1× to 20×; the PDI went from 0.17 to 0.06 for sucrose compositions, from 0.40 to 0.18 for dextran compositions, and from 0.15 to 0.10 for trehalose dihydrate compositions (FIG. 8F).

5—Zeta Potential Measurement of Concentrated Compositions Containing 3.44 mM Histidine, Rehydrated to Higher Concentrations

Duplicates of composition #15 were prepared Rh1× and six replicates of each of the other compositions (#17 to 19) were prepared (two fresh, two Rh1×, and two Rh20×) as described in Section 3. Rehydrated samples were left to stabilize for 15 to 30 minutes. All samples rehydrated within 5 minutes, although Rh20× was harder to achieve given the small rehydration volume relative to the cake volume. Fresh samples and rehydrated samples were supplemented with 600 μL 13 mM NaCl and, if necessary, their volume was completed to 800 μL with Milli-Q prior to Zeta potential analysis. Zeta potential was measured as described in Example 2. Freshly prepared compositions had zeta potentials of 24 mV; freeze-dried and rehydrated compositions had zeta potentials of 18 to 21 mV, independently of their lyoprotectant or rehydration volume (FIG. 8G).

6—ESEM Imaging of Concentrated Compositions Containing 3.44 mM Histidine, Rehydrated to Higher Concentrations

Six replicates of each of the other compositions (#17 to 19) were prepared as described in Section 3: two Rh1×, two Rh10×, and two Rh20×.ESEM sample preparation and imaging was performed as described in Example 1. Nanoparticles observed for compositions containing 3.44 mM histidine were less spherical in shape than previously observed for compositions containing 13.75 mM histidine, which is consistent with the variations in PDIs observed in DLS. No significant difference was observed between compositions that were freeze-dried and then Rh1× or Rh20×, although they seemed to have more spherical particles than freshly prepared compositions. Particles were mostly inferior to 200 nm in diameter (data not shown).

7—In Vitro Transfection

Six replicates of each composition (#15 to 22) were prepared as described in Section 3: two fresh, two Rh1×, and two Rh20×. Fugene-based lipoplexes were used as positive controls for transfection efficiency. In vitro transfection was performed as described in Example 1.

8—pH

The pH of compositions #15 to 22 was measured in freshly prepared samples and in samples freeze-dried and rehydration to their initial volume (Rh1×) or to one twentieth their initial volume (Rh20×). In absence of L-histidine, freshly prepared samples had an average pH of 5.8±0.2, independently of the presence or nature of the lyoprotectant. Their average pH was 7.0±0.2 following Rh1× and 5.1±0.2 following Rh20×. Freshly prepared compositions containing 3.44 mM L-histidine had an average pH of 6.42±0.05, independently of the presence or nature of the lyoprotectant, whereas pH of freeze-dried samples was 6.50±0.06 after Rh1× and 6.48±0.02 after Rh20×.

9—Osmolality

Since the above method yields compositions with twice the amount of complexes, validity of the osmolality model previously developed was verified for compositions #17 to 19, freeze-dried and rehydrated 5 times more concentrated. The model was acceptable for compositions containing sucrose (#17) or trehalose (#19), with osmolality underestimations of 6 and 8% respectively, but was inadequate for those containing dextran (#18), with an underestimation of the osmolality of 57%. Osmolalities of compositions #17 and 19 were estimated for fresh or freeze-dried samples rehydrated to their initial volume (Rh1×), to one tenth their initial volumes (Rh10×) and to one twentieth their initial volumes (Rh20×). Based on the model, osmolalities varied between 19 and 372 mOsm for samples containing sucrose, and between 17 and 339 mOsm for samples containing trehalose dihydrate. Both compositions Rh20× were close to isotonicity: 0.5% suc−his(3.44)−Rh20× at 372 mOsm, and 0.5% tre−his(3.44)−Rh20× at 339 mOsm.

10—Transfection Efficiency

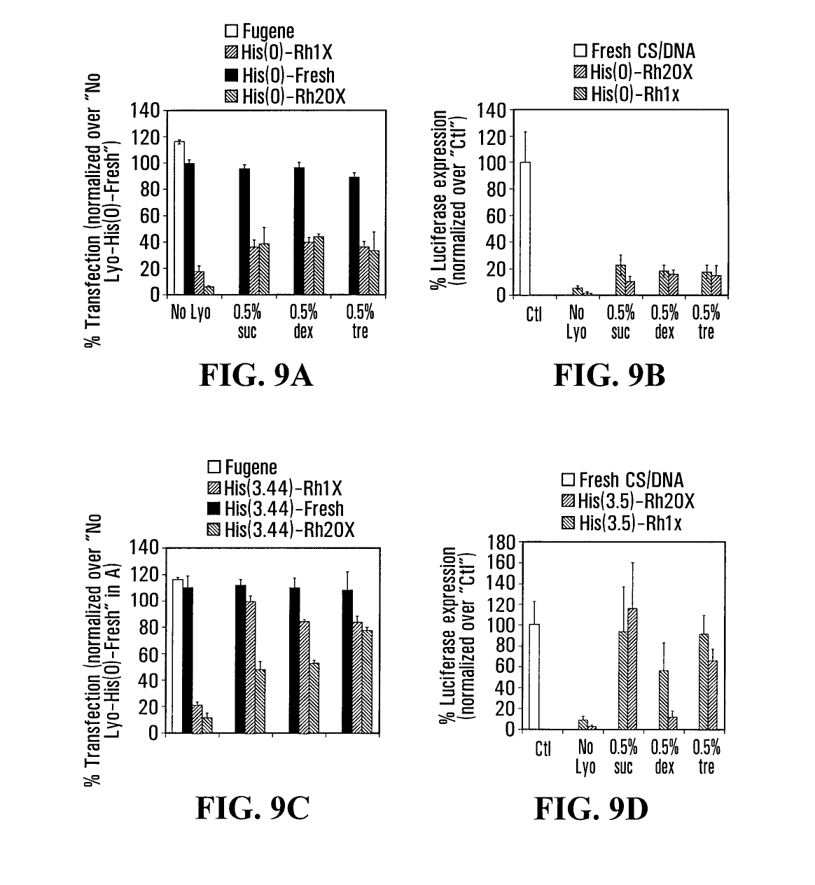

Transfection efficiency was measured as described in Example 1. Sample transfection efficiencies were normalized over the value obtained for fresh complexes in absence of excipients (FIG. 9A, A: No Lyo-His(0)-Fresh), which had a transfection efficiency of 53% of total cells. Fugene had a transfection efficiency of 116% of the fresh control (FIGS. 9A and 9C). Fresh compositions without histidine had transfection efficiencies of 90 to 100% of fresh control (FIG. 9A); fresh compositions with 3.44 mM histidine had transfection efficiencies of 108 to 113% of fresh control (FIG. 9C). Compositions freeze-dried in absence of lyoprotectant, with or without 3.44 mM histidine, had transfections efficiencies below 22% of control (FIGS. 9A and 9C). Compositions freeze-dried with 0.5% (w/v) lyoprotectant, but without histidine, had transfection efficiencies around 40% of control (FIG. 9A). Compositions freeze-dried with 0.5% (w/v) lyoprotectant and 3.44 mM histidine, and rehydrated 1× (Rh1×), had transfection efficiencies, relative to the fresh control, of: 100% for sucrose, 85% for dextran, and 83% for trehalose (FIG. 9C). Compositions freeze-dried with 0.5% (w/v) lyoprotectant and 3.44 mM histidine, and rehydrated 20× (Rh20×), had transfection efficiencies, relative to the fresh control, of: 48% for sucrose, 53% for dextran, and 78% for trehalose (FIG. 9C).

11—Luciferase Expression

Luciferase expression was quantified as described in Example 1. Luciferase relative light units per minute (RLU/min) measured were converted to μM using a standard curve made of serial dilutions of a recombinant luciferase standard of know concentration. Sample luciferase expression levels were normalized over the value obtained for fresh chitosan/DNA complexes in absence of excipients (Ctl), which had a expression level of 6.76E-5 μM of luciferase/mg of proteins. Compositions freeze-dried in absence of lyoprotectant, with or without 3.44 mM histidine, expressed less than 10% of the luciferase level measured for the control (FIGS. 9B and 9D). Compositions freeze-dried with 0.5% (w/v) lyoprotectant, but without histidine, expressed less than 25% of the luciferase level measured for the control (FIG. 9B). Compositions freeze-dried with 0.5% (w/v) lyoprotectant and 3.44 mM histidine, and rehydrated 1× (Rh1×), had luciferase expression levels similar to the positive control for sucrose and trehalose dihydrate, and 56% that of the positive control for dextran (FIG. 9D). Compositions freeze-dried with 0.5% (w/v) lyoprotectant and 3.44 mM histidine, and rehydrated 20× (Rh20×), had luciferase expression levels similar to the positive control for sucrose, 12% that of the positive control for dextran, and 65% that of the positive control for trehalose dihydrate (FIG. 9D).

TABLE 17
Performance of the 22 different compositions.

| Criteria | | |
|---|---|---|
| number | Criteria description | Performance |
| 1 | The nanoparticle Z-average should be below 250 | Passed: #1-14, 17, 18 (Rh1 & 10X), 19 |
| | nm (Assessed by DLS) | Failed: #18 (Rh20X) |
| | | Not checked: 20-22 |
| 2 | The nanoparticle average PDI should be at most | Passed: #1-8, 10-12, 14-17, 18 |
| | 0.25 (Assessed by DLS) | (Rh10X, Rh20X), 19 |
| | | Failed: #9, 13, 18 (Rh1X) |
| | | Not checked: 20-22 |
| 3 | The nanoparticle average zeta potential should be | Passed: 15, 17-19 |
| | positive and sufficient to ensure composition short- | Not checked: others |
| | term stability (Assessed by LDV) | |
| 4 | There should be no aggregation in the samples | Passed: 17-19 |
| | (Assessed by ESEM) | Not checked: others |
| 5 | The composition transfection level should be | Passed: |
| | greater than 50% of the transfection level of fresh | Fresh: 15-22 |
| | CS/DNA particles without excipients | Rh1X: 17-19 |
| | (Assessed by flow cytometry) | Rh20X: 19 |
| | | Failed: |
| | | Rh1X: 15-16, 20-22 |
| | | Rh20X: 15-18, 20-22 |
| | | Not checked: 1-14 |
| 6 | The composition luciferase expression level should | Passed: |
| | be greater than 50% of the expression level of | Rh1X: 17-19 |
| | fresh CS/DNA particles without excipients | Rh20X: 17, 19 |
| | (Assessed by luminometry) | Failed: |
| | | Rh1X: 15-16, 20-22 |
| | | Rh20X:15, 16, 18, 20-22 |
| | | Not checked: 1-14, 15-22(fresh) |
| 7 | The freeze-dried cake should be completely | Passed: all |
| | reconstituted within 5 minutes | |
| | (Assessed with visual inspection upon | |
| | reconstitution) | |
| 8 | The final DNA concentration should be at least 0.5 | Passed: 17-22, when Rh10X or 20X |
| | mg/mL | (concentrations at 0.5 mg/ml after Rh10X |
| | (Assessed from the initial DNA content and the | and at 1 mg/ml after Rh20X) |
| | rehydration factor used) | Failed: others (concentrations at 0.05 |
| | | mg/ml fresh or after Rh1X) |
| 9 | The rehydrated compositions should be near iso- | Passed: #17(Rh20X), 19(Rh20X) |
| | osmolality: between 200 and 400 mOsm | Not checked: #18 |
| | (Assessed with the osmolality model of the | |
| | compositions) | |
| 10 | The rehydrated compositions should have a near- | Passed: |
| | netrual pH: between 6 and 7 | Fresh: 16-19 |
| | (Assessed with a pH meter) | Rh1X: 15-22 |
| | | Rh20X: 16-19 |
| | | Failed: #15, 20-22 (fresh and Rh20X) |

### Example 7

Compositions can be concentrated up to 20 fold using two successive freeze-drying/rehydration cycles, so that final rehydration prior to injection is facilitated (higher rehydration volume to cake volume), this without significant changes to nanoparticle physico-chemical properties and transfection efficiency

1—Preparation of Chitosan/DNA Nanoparticle Compositions Containing 0.5% (w/v) Trehalose Dihydrate and 3.5 mM Histidine for Multiple Freeze-Drying

Chitosan (Mn 10 kDa, 92% DDA) was dissolved in HCl overnight at room temperature to obtain a final chitosan concentration of 5 mg/mL. The chitosan stock solution was diluted to 271 μg/ml using sterile 2% (w/v) trehalose dihydrate, sterile 14 mM L-histidine buffer at pH 6.5, and Milli-Q water, as per Table 18.

TABLE 18
Chitosan dilution with excipients prior to complex formation.

| | Volume (μL) | | | |
|---|---|---|---|---|
| | | | 14 mM | |
| | Chitosan | 2% (w/v) | L-histidine | Milli-Q |
| Composition | 5 mg/mL | Trehalose | pH 6.5 | water |
| 0.5% tre-his(3.5) | 711.5 | 3282 | 3282 | 5852.5 |

DNA (pEGFPLuc at 400 μg/mL) stock solution was diluted to 100 μg/ml using sterile 2% (w/v) trehalose dihydrate, sterile 14 mM L-histidine buffer at pH 6.5, and Milli-Q water, as per Table 19.

TABLE 19
DNA dilution with excipients prior to complex formation.

| | Volume (μL) | | | |
|---|---|---|---|---|
| | | | 14 mM | |
| | DNA | 2% (w/v) | L-histidine | Milli-Q |
| Composition | 400 μg/mL | Trehalose | pH 6.5 | water |
| 0.5% tre-his(3.5) | 3282 | 3282 | 3282 | 3282 |

21 samples were prepared. For each sample, 625 μL of diluted chitosan solution (Table 18) was mixed with 625 μL of diluted DNA solution (Table 19), in order to form complexes at N/P ratio of 5. Mixing was performed by pipetting the solution up and down approximately 10 times immediately following addition of chitosan. Samples were left to stabilize at room temperature for 30 minutes prior to analysis or freeze-drying.

2—Sample Freeze-Drying

15 samples were freeze-dried. For each sample, 1200 μL was transferred to a 10 mL serum vial, as per Table 20, and freeze-dried with 20 mm butyl lyophilization stoppers, as described in Example 2. 6 samples were rehydrated Rh10× with 120 μL, and then 100 μL of each of sample was transferred to a 2 mL serum vial, as per Table 20, and freeze-dried with 13 mm butyl lyophilization stoppers, as described in Example 2. 3 samples were rehydrated Rh5× with 240 μL, and then 200 μL was used to fill two 2 mL serum vials with 100 μL of sample: one vial for DLS analysis, the other for transfection, as per Table 20. Samples were freeze-dried with 13 mm butyl lyophilization stoppers, as described in Example 2.

TABLE 20
Chitosan dilution with excipients prior to complex formation.

| | | 1st Freeze-Drying | | | | 2nd Freeze-Drying | | | | |
|---|---|---|---|---|---|---|---|---|---|---|
| | | | | Rehydration | | | | Rehydration | | Final |
| Sample | | # 10 mL | FD vol | Vol | Conc. | # 2 mL | FD vol | Vol | Conc. | conc. |
| # | Label | vials | (μL) | (μL) | factor | vials | (μL) | (μL) | factor | factor |
| 1 | FD/Rh20X | 6 | 1200 | 60 | 20X | NA | | | | 20X |
| 2 | Rh(10X + 2X) | 6 | 1200 | 120 | 10X | 6 | 100 | 50 | 2X | 20X |
| 3 | Rh(5X + 4X) | 3 | 1200 | 240 | 5X | 6 | 100 | 25 | 4X | 20X |

3—Rehydration of Samples for DLS or Transfection

Experimentation showed that dilution of rehydrated samples has no impact on nanoparticle properties (size, Zeta potential, transfection efficiency, etc.), therefore samples were rehydrated and diluted as follows prior to analysis.

Samples #1 were rehydrated Rh20× with 60 μL Milli-Q 30 min prior to analysis, and then diluted with 1140 μL Milli-Q 15 min prior to analysis.

Samples #2 were rehydrated Rh20× (10×+2×) with 50 μL Milli-Q 30 min prior to analysis, and then diluted with 950 μL Milli-Q 15 min prior to analysis.

Samples #3 were rehydrated Rh20× (5×+4×) with 25 μL Milli-Q 30 min prior to analysis, and then diluted with 475 μL Milli-Q 15 min prior to analysis.

All samples rehydrated within 5 minutes, although sample #1 (Rh20×) were harder to achieve given the small rehydration volume relative to the cake volume. Samples #2 and 3 were easy and quick to rehydrate, while also reaching a final concentration factor of 20×.

4—DLS Measurements of Concentrated Compositions Containing 0.5% (w/v) Trehalose Dihydrate and 3.5 mM Histidine, Rehydrated to Higher Concentrations

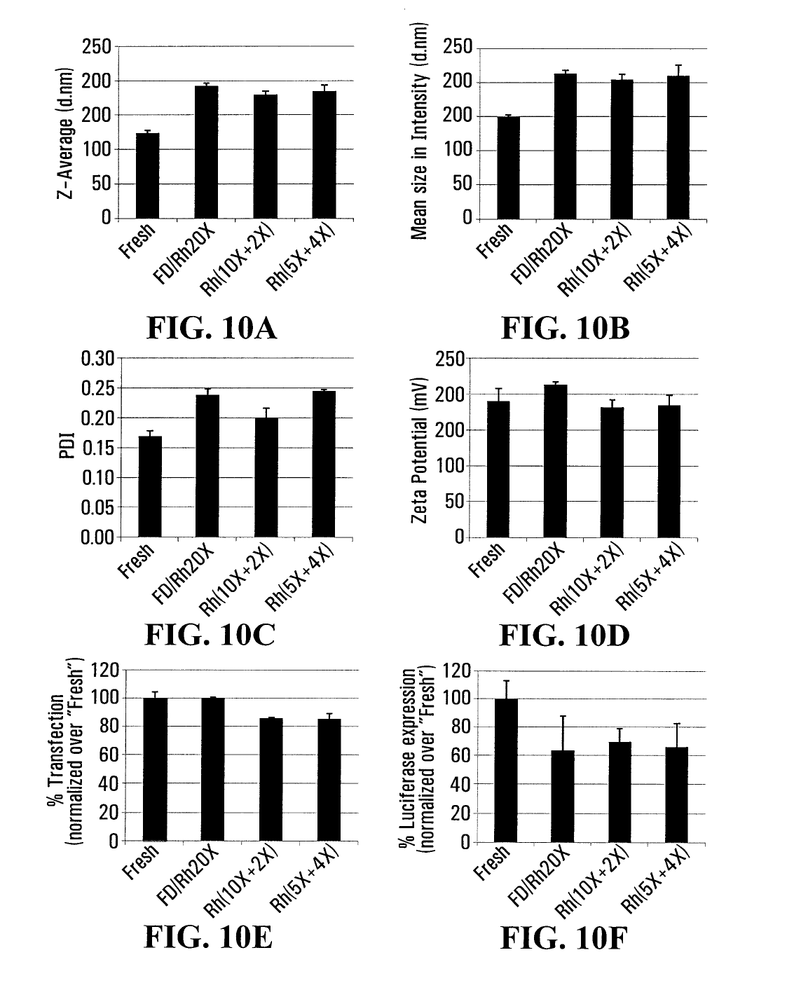

Three replicates were freshly prepared as described in Section 1, and three freeze-dried replicates of each composition were rehydrated as described in Section 3. Size and PDI analyses were done as described in Example 2. Nanoparticles formulated in 0.5% (w/v) trehalose dihydrate and 3.5 mM L-histidine could be freeze-dried twice to reach the final concentration factor of 20× (Rh20×) without seeing particle aggregation. Compared to freshly prepared particles, Z-averages increased by 56 to 68 nm and mean sizes in intensity increased by 54 to 63 nm, depending on the number of freeze-drying and rehydration cycles used to reach Rh20×. Z-averages (180 to 192 nm) and mean sizes in intensity (204 to 213 nm) were similar between samples Rh20×, independently of the number of freeze-drying and rehydration cycles performed (FIGS. 10A-B). PDI values increased slightly following freeze-drying and rehydration, from 0.17, when freshly prepared, to between 0.20 and 0.25 after Rh20×. (FIG. 10C).

5—Zeta Potential Measurement of Concentrated Compositions Containing 0.5% (w/v) Trehalose Dihydrate and 3.5 mM Histidine, Rehydrated to Higher Concentrations

Samples previously analyzed by DLS (Section 4) were supplemented with 400 μL 20 mM NaCl, then their zeta potential was measured as described in Example 2. Freshly prepared nanoparticles had an average zeta potential of 19 mV; freeze-dried and rehydrated compositions had zeta potentials of 18 to 21 mV, independently of the number of freeze-drying cycles used to reach Rh20× (FIG. 10D).

6—In Vitro Transfection of Concentrated Compositions Containing 0.5% (w/v) Trehalose Dihydrate and 3.5 mM Histidine, Rehydrated to Higher Concentrations

Three replicates were freshly prepared as described in Section 1, and three freeze-dried replicates of each composition were rehydrated as described in Section 3. In vitro transfection was performed as described in Example 1.

7—Transfection Efficiency of Concentrated Compositions Containing 0.5% (w/v) Trehalose Dihydrate and 3.5 mM Histidine, Rehydrated to Higher Concentrations

Transfection efficiency was measured as described in Example 1. Sample transfection efficiencies were normalized over the value obtained for fresh complexes prepared in 0.5% (w/v) trehalose dihydrate and 3.5 mM L-histidine at pH6.5 (FIG. 10E: Fresh), which had a transfection efficiency of 44% of total cells. All freeze-dried compositions had transfection efficiencies of 85 to 100% of fresh control (FIG. 10E): compositions rehydrated 20× after a single freeze-drying cycle (FD/Rh20×) had a transfection efficiency equal (100%) to the fresh samples; compositions rehydrated 10×, then freeze-dried and Rh2× [Rh(10×+2×)], had transfections efficiencies of 86% of control; and compositions rehydrated 5×, then freeze-dried and Rh4× [Rh(5×+4×)], had transfections efficiencies of 85% of control.

8—Luciferase Expression of Concentrated Compositions Containing 0.5% (w/v) Trehalose Dihydrate and 3.5 mM Histidine, Rehydrated to Higher Concentrations

Luciferase expression was quantified as described in Example 1, and expressed in relative light units per minute (RLU/min). Sample luciferase expression levels were normalized over the value obtained for fresh complexes prepared in 0.5% (w/v) trehalose dihydrate and 3.5 mM L-histidine at pH6.5 (FIG. 10F: Fresh), which had an expression level of 5.24E+8 RLU/min*mg of proteins. All freeze-dried compositions with final Rh20× had similar luciferase expression levels, with values of 64 to 69% of that of fresh control (FIG. 10F): compositions rehydrated 20× after a single freeze-drying cycle (FD/Rh20×) had a luciferase expression level of 64% of control; compositions rehydrated 10×, then freeze-dried and Rh2× [Rh(10×+2×)], had a higher luciferase expression levels, with 69% of the expression of control; and compositions rehydrated 5×, then freeze-dried and Rh4× [Rh(5×+4×)], had luciferase expression levels of 66% of control.

TABLE 21
Performance of the 5 different compositions.

| Criteria | | |
|---|---|---|
| number | Criteria description | Performance |
| 1 | The nanoparticle Z-average should be below 250 nm (Assessed by DLS) | Passed: all |
| 2 | The nanoparticle average PDI should be below 0.25 (Assessed by DLS) | Passed: all |
| 3 | The nanoparticle average zeta potential should be positive and sufficient to ensure | Passed: all |
| | composition short-term stability (Assessed by LDV) | |
| 4 | There should be no aggregation in the samples (Assessed by ESEM) | Passed: all |
| 5 | The composition transfection level should be greater than 50% of the transfection level of | Passed: all |
| | fresh CS/DNA particles (Assessed by flow cytometry) | |
| 6 | The composition luciferase expression level should be greater than 50% of the expression | Passed: all |
| | level of fresh CS/DNA particles (Assessed by luminometry) | |
| 7 | The freeze-dried cake should be completely reconstituted within 5 minutes | Passed: all |
| | (Assessed with visual inspection upon reconstitution) | |
| 8 | The final DNA concentration should be at least 0.5 mg/mL | Passed: all |
| | (Assessed from the initial DNA content and the rehydration factor used) | |
| 9 | The rehydrated compositions should be near iso-osmolality: between 200 and 400 mOsm | Passed: all |
| | (Assessed with the osmolality model of the compositions) | |
| 10 | The rehydrated compositions should have a near-netrual pH: between 6 and 7 | Passed: all |
| | (Assessed with a pH meter) | |

### Example 8

Chitosan/siRNA nanoparticles can be prepared at higher initial nucleic acid concentration, as compared to CS/DNA nanoparticle, but excipient content must be increased accordingly;

These compositions can be concentrated up to 10 fold without significant changes to nanoparticle physico-chemical properties and silencing efficiency.

1—Preparation of Concentrated Chitosan/siRNA Nanoparticle Compositions

Chitosan (Mn 10 kDa, 92% DDA) was dissolved in HCl overnight at room temperature to obtain a final chitosan concentration of 5 mg/mL. The chitosan stock solution was diluted to 271 or 542 μg/ml using sterile lyoprotectant solutions (8% (w/v) dextran 5 kDa or trehalose dihydrate), sterile 14 mM L-histidine buffer at pH 6.5 and RNase-free water, as per Table 22.

TABLE 22
Chitosan dilution with excipients prior to complex formation.

| | | Volume added in each sample (μL) | | | | |
|---|---|---|---|---|---|---|
| | | | L-histidine | | | |
| Sample | | CS | pH6.5 | Tre | Dex | RNAse-free |
| # | Description | 5 mg/mL | 14 mM | 8% | 8% | H₂O |
| 1 | 0.5% tre-his(3.5)—100 μg | 24 | 55 | 13.8 | 0 | 127.2 |
| 2 | 1% tre-his(7)—100 μg | 24 | 110 | 27.6 | 0 | 58.6 |
| 3 | 1% tre-his(3.5)—100 μg | 24 | 55 | 27.6 | 0 | 113.6 |
| 4 | 2% tre-his(3.5)—100 μg | 24 | 55 | 55 | 0 | 86 |
| 5 | 0.5% tre-0.5% dex-his(3.5)—100 μg | 24 | 55 | 13.8 | 13.8 | 113.6 |
| 6 | 0.5% tre-1% dex-his(3.5)—100 μg | 24 | 55 | 13.8 | 27.6 | 99.8 |
| 7 | 0.5% tre-his(3.5)—50 μg | 12 | 55 | 13.8 | 0 | 139.2 |

Anti-ApoB siRNA (sense: GUCAUCACACUGAAUACCAAU, antisense: AUUGGUAUUCAGUGUGAUGACAC, at 1 mg/mL) stock solution was diluted to 100 or 200 μg/ml using sterile lyoprotectant solutions (8% (w/v) dextran 5 kDa or trehalose dihydrate), 14 mM L-histidine buffer at pH 6.5 and/or RNase-free water, as per Table 23.

TABLE 23
siRNA dilution with excipients prior to complex formation.

| | | Volume added in each sample (μL) | | | | |
|---|---|---|---|---|---|---|
| | | | L-histidine | | | |
| Sample | | siRNA | pH6.5 | Tre | Dex | RNAse-free |
| # | Description | 1 mg/mL | 14 mM | 8% | 8% | H₂O |
| 1 | 0.5% tre-his(3.5)—100 μg | 44 | 55 | 13.8 | 0 | 107.2 |
| 2 | 1% tre-his(7)—100 μg | 44 | 110 | 27.6 | 0 | 38.6 |
| 3 | 1% tre-his(3.5)—100 μg | 44 | 55 | 27.6 | 0 | 93.6 |
| 4 | 2% tre-his(3.5)—100 μg | 44 | 55 | 55 | 0 | 66 |
| 5 | 0.5% tre-0.5% dex-his(3.5)—100 μg | 44 | 55 | 13.8 | 13.8 | 93.6 |
| 6 | 0.5% tre-1% dex-his(3.5)—100 μg | 44 | 55 | 13.8 | 27.6 | 79.8 |
| 7 | 0.5% tre-his(3.5)—50 μg | 22 | 55 | 13.8 | 0 | 129.2 |

For each replicate, 100 μL of chitosan solution was mixed with 100 μL of its complementary siRNA solution (for example, chitosan Composition #1 with siRNA Composition #1), in order to form complexes at N/P ratio of 5. Mixing was performed by pipetting the solution up and down approximately 10 times immediately following addition of chitosan. Samples were left to stabilize at room temperature for 30 minutes prior to analysis.

2—Freeze-Drying of Concentrated Chitosan/siRNA Nanoparticle Compositions

Samples to be freeze-dried were transferred to 2 mL serum vials and freeze-dried with 13 mm butyl lyophilization stoppers. A water permeable membrane was placed over the tray containing all samples to prevent dust or bacterial contamination. Freeze-drying was carried in a Millrock Laboratory Series Freeze-Dryer PC/PLC, using the following cycle: step cool to 5° C. and maintain isothermal for 30 min, step cool to −5° C. and maintain isothermal for 30 min, then ramp freeze to −40° C. in 35 min and maintain isothermal for 2 h; primary drying for 48 hours at −40° C., at 100 millitorrs; and secondary drying at 100 millitorrs, increasing temperature to 30° C. in 12 hours and then maintaining isothermal at 30° C. for 6 hours. Samples were stoppered, crimped and stored at 4° C. until use. 15 to 30 minutes prior to use, Rh1× samples were rehydrated with 200 μL of RNase-free water, Rh10× samples were rehydrated with 20 μL of RNase-free water, and Rh20× samples were rehydrated with 10 μL of RNase-free water. All samples rehydrated within 5 minutes.

4—DLS Measurements of Concentrated Chitosan/siRNA Nanoparticle Compositions

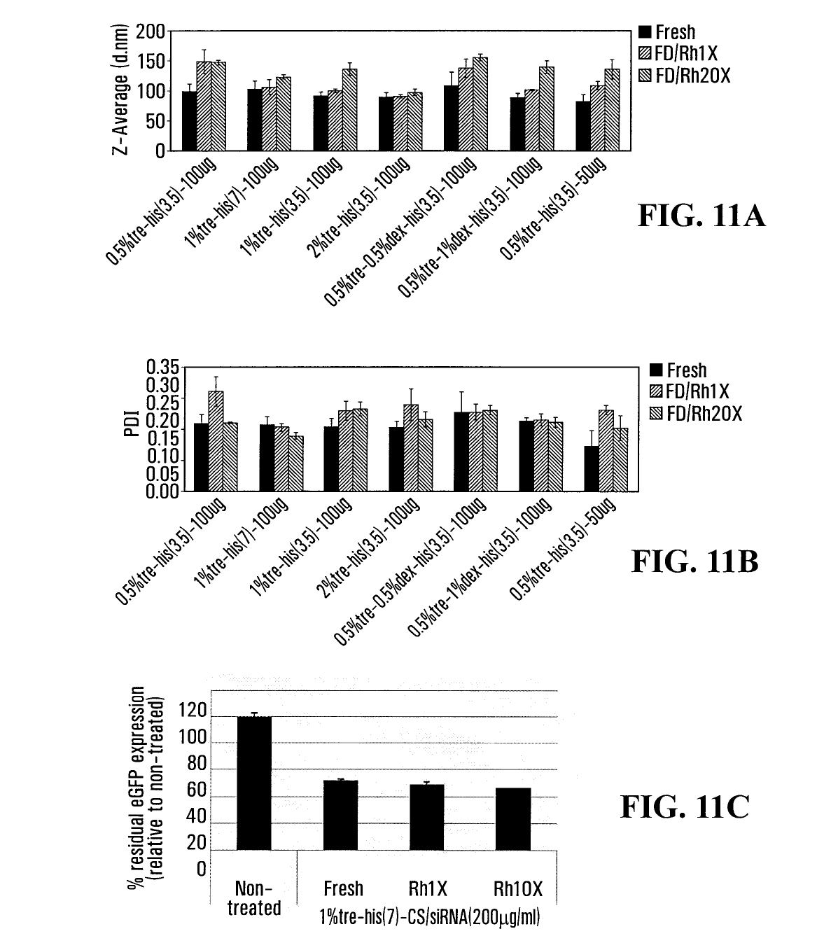

Nine replicates of each composition were prepared as described in Sections 1 and 2: three freshly prepared (no FD), three Rh1×, and three Rh20×. Size and PDI analyses were done as described in Example 2. Although all compositions prevented severe aggregation after Rh20×, only compositions #2 and 4 showed no significant change in particle size after Rh1× and/or Rh20×, as compared to fresh compositions; their Z-averages increased by 21 and 9 nm respectively (FIG. 11A). Average PDI values were mostly below 0.25 (except composition #1, Rh1×), with compositions #2 and 4 having PDI values of 0.16 and 0.20 respectively after Rh20× (FIG. 11B).

5—Zeta Potential Measurement of Concentrated Chitosan/siRNA Nanoparticle Compositions

Nine replicates of each composition were prepared as described in Sections 1 and 2: three freshly prepared (no FD), three Rh1×, and three Rh10×. Rehydrated samples were left to stabilize for 15 to 30 minutes, though all were rehydrated within 5 minutes. Volumes of fresh and rehydrated samples were brought to 400 μL using RNase-free water, then 400 μL 20 mM NaCl was added prior to Zeta potential analysis. Zeta potential was measured as described in Example 2. Freshly prepared compositions had zeta potentials of 21 mV; freeze-dried and rehydrated compositions had zeta potentials of 21 to 23 mV, independently of their rehydration volume.

6—ESEM Imaging of Concentrated Chitosan/siRNA Nanoparticle Compositions

Nine replicates of composition #2 were prepared as described in Sections 1 and 2: three freshly prepared (no FD), three Rh1×, and three Rh10×. ESEM sample preparation and imaging was performed as described in Example 1. All nanoparticles observed were spherical in shape and were mostly inferior to 100 nm in diameter. No significant difference was observed between particles from fresh, Rh1× or Rh10× compositions.

7—In Vitro Silencing of Concentrated Chitosan/siRNA Nanoparticle Compositions

Nine replicates of composition #2 were prepared as described in Sections 1 and 2: three freshly prepared (no FD), three Rh1×, and three Rh20×. DharmaFECT2 was used as positive controls for silencing efficiency. eGFP positive H1299 cells, grown in RPMI-1640 at pH7.2, supplemented with 10% fetal bovine serum (FBS), and incubated at 37° C. in 5% CO₂, were used for in vitro studies. 45,000 cells were plated per well of a 24-well plate 24 hours prior to transfection in order to reach about 75-85% confluency for transfection. In each well, the culture medium was replaced with DMEM high glucose at pH6.5 (without FBS) and nanoparticle composition, for a total of 500 μL of solution containing 100 nM of siRNA. Cells were incubated 4 hours at 37° C., in 5% CO₂, then supplemented with 55 μL FBS, and then incubated another 44 h prior to analysis.

8—Silencing Efficiency of Concentrated Chitosan/siRNA Nanoparticle Compositions

Silencing efficiency was measured using flow cytometry. Sample preparation: growth medium was removed from each well containing a sample to be analyzed; cells were washed with 500 μL phosphate buffered saline (PBS) at pH 7.4; they were trypsinized 5 minutes at 37° C. using 75 μL trypsin/EDTA per well; then 325 μL growth medium was added and the whole sample was transferred into a cytometry tube. Flow cytometry measurements: 10000 events were collected per sample, and the mean fluorescence intensity was measured through 510/20 nm bandpass filters with photomultiplier tubes following excitation of enhanced green fluorescence protein (EGFP) in cells using a 488 nm argon laser. Forward scatter (FSC) and side scatter (SSC) were also used to exclude dead cells and debris from events recorded. Finally, FSC was used to identify and exclude doublets from the events when analyzing transfection efficiency using the data. The mean residual eGFP intensity was expressed as a percentage of the mean eGFP expression measured for non-treated cells. Although silencing efficiency of composition #2 was lower than DharmaFECT2, with residual eGFP expression of 5% (data not shown), FD had no negative impact on the silencing efficiency of CS/siRNA. Fresh compositions had residual eGFP expression of 52% of untreated cells; Rh1×, of 49%; and Rh10×, of 47% (FIG. 11C).

TABLE 24
Performance of the different compositions.

| Criteria | | |
|---|---|---|
| number | Criteria description | Performance |
| 1 | The nanoparticle Z-average should be below 250 | Passed: all. |
| | nm (Assessed by DLS) | Failed: none. |
| 2 | The nanoparticle average PDI should be at most | Passed: #1 (Fresh & Rh20X), 2-7 |
| | 0.25 (Assessed by DLS) | Failed: #1 (Rh1X) |
| 3 | The nanoparticle average zeta potential should be | Passed: 2 |
| | positive and sufficient to ensure composition short- | Not checked: others |
| | term stability (Assessed by LDV) | |
| 4 | There should be no aggregation in the samples | Passed: 2 |
| | (Assessed by ESEM) | Not checked: others |
| 5 | The composition silencing efficiency should be | Passed: 2 |
| | greater than 50% of the silencing efficiency of fresh | No checked: others. |
| | CS/siRNA particles (Assessed by flow cytometry) | |
| 6 | The freeze-dried cake should be completely | Passed: all |
| | reconstituted within 5 minutes | |
| | (Assessed with visual inspection upon | |
| | reconstitution) | |
| 7 | The final DNA concentration should be at least 0.5 | Passed: |
| | mg/mL | #1, 5, 6: 2 mg/mL after Rh20X |
| | (Assessed from the initial DNA content and the | #2, 3: 1 mg/mL after Rh10X |
| | rehydration factor used) | #4: 0.5 mg/mL after Rh5X |
| | | #7: 1 mg/mL after Rh20X |
| | | Failed: none. |
| 9 | The rehydrated compositions should be near iso- | Passed: #1(Rh20X), 2 (Rh10X), 3 (Rh10X), |
| | osmolality: between 200 and 400 mOsm | 4 (Rh5X), 5 (Rh20X), 6 (Rh20X), 7 (Rh20X) |
| | (Assessed with the osmolality model of the | Failed: #2-4 at Rh20X |
| | compositions) | |
| 10 | The rehydrated compositions should have a near- | Passed: all. |
| | netrual pH: between 6 and 7 | Failed: none. |
| | (Assessed with a pH meter) | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All documents mentioned in the specification are herein incorporated by reference.

### REFERENCES

1. Xu, L. and T. Anchordoquy, Drug delivery trends in clinical trials and translational medicine: Challenges and opportunities in the delivery of nucleic acid-based therapeutics. Journal of Pharmaceutical Sciences, 2011. 100(1): p. 38-52.
2. Tang, X. L. and M. J. Pikal, Design of freeze-drying processes for pharmaceuticals: Practical advice. Pharmaceutical Research, 2004. 21(2): p. 191-200.
3. Wang, W., Lyophilization and development of solid protein pharmaceuticals. International Journal of Pharmaceutics, 2000. 203(1-2): p. 1-60.
4. Abdelwahed, W., et al., Freeze-drying of nanoparticles: Formulation, process and storage considerations. Advanced Drug Delivery Reviews, 2006. 58(15): p. 1688-1713.
5. Anchordoquy, T. J., et al., Physical stabilization of DNA-based therapeutics. Drug Discovery Today, 2001. 6(9): p. 463-470.
6. Schwarz, C. and W. Mehnert, Freeze-drying of drug-free and drug-loaded solid lipid nanoparticles (SLN). International Journal of Pharmaceutics, 1997. 157(2): p. 171-179.
7. Hinrichs, W. L. J., et al., The choice of a suitable oligosaccharide to prevent aggregation of PEGylated nanoparticles during freeze thawing and freeze drying. International Journal of Pharmaceutics, 2006. 311(1-2): p. 237-244.
8. Mao, H.-Q., et al., Chitosan-DNA nanoparticles as gene carriers: synthesis, characterization and transfection efficiency. Journal of Controlled Release, 2001. 70(3): p. 399-421.
9. Andersen, M. Ø., et al., Delivery of siRNA from lyophilized polymeric surfaces. Biomaterials, 2008. 29(4): p. 506-512.
10. Tahara, K., et al., Establishing chitosan coated PLGA nanosphere platform loaded with wide variety of nucleic acid by complexation with cationic compound for gene delivery. International Journal of Pharmaceutics, 2008. 354(1-2): p. 210-216.
11. Anchordoquy, T. J. and G. S. Koe, Physical stability of nonviral plasmid-based therapeutics. Journal of Pharmaceutical Sciences, 2000. 89(3): p. 289-296.
12. Liu, J. S., Physical characterization of pharmaceutical formulations in frozen and freeze-dried solid states: Techniques and applications in freeze-drying development. Pharmaceutical Development and Technology, 2006. 11(1): p. 3-28.
13. Schersch, K., et al., Systematic investigation of the effect of lyophilizate collapse on pharmaceutically relevant proteins I: Stability after freeze-drying. Journal of Pharmaceutical Sciences, 2009. 99(5): p. 2256-2278.
14. Hinrichs, W. L. J., M. G. Prinsen, and H. W. Frijlink, Inulin glasses for the stabilization of therapeutic proteins. International Journal of Pharmaceutics, 2001. 215(1-2): p. 163-174.
15. Anchordoquy, T. J., T. K. Armstrong, and M. d. C. Molina, Low molecular weight dextrans stabilize nonviral vectors during lyophilization at low osmolalities: concentrating suspensions by rehydration to reduced volumes. Journal of Pharmaceutical Sciences, 2005. 94(6): p. 1226-1236.
16. Allison, S. D. and T. J. Anchordoquy, Mechanisms of protection of cationic lipid-DNA complexes during lyophilization. Journal of Pharmaceutical Sciences, 2000. 89(5): p. 682-691.
17. Hinrichs, W. L. J., et al., Inulin is a promising cryo-and lyoprotectant for PEGylated lipoplexes. Journal of Controlled Release, 2005. 103(2): p. 465-479.
18. V\dot{a}rum, K. M., M. H. Ottøy, and O. Smidsrød, Acid hydrolysis of chitosans. Carbohydrate Polymers, 2001. 46(1): p. 89-98.
19. Chen, R. H., et al., Changes in the Mark-Houwink hydrodynamic volume of chitosan molecules in solutions of different organic acids, at different temperatures and ionic strengths. Carbohydrate Polymers, 2009. 78(4): p. 902-907.
20. Gómez, G., M. J. Pikal, and N. Rodríguez-Hornedo, Effect of Initial Buffer Composition on pH Changes During Far-From-Equilibrium Freezing of Sodium Phosphate Buffer Solutions. Pharmaceutical Research, 2001. 18(1): p. 90-97.
21. Sundaramurthi, P., E. Shalaev, and R. Suryanarayanan, Calorimetric and Diffractometric Evidence for the Sequential Crystallization of Buffer Components and the Consequential pH Swing in Frozen Solutions. Journal of Physical Chemistry B, 2010. 114(14): p. 4915-4923.
22. Shalaev, E. Y., et al., Thermophysical properties of pharmaceutically compatible buffers at sub-zero temperatures: Implications for freeze-drying. Pharmaceutical Research, 2002. 19(2): p. 195-201.
23. Bhatnagar, B. S., R. H. Bogner, and M. J. Pikal, Protein Stability During Freezing: Separation of Stresses and Mechanisms of Protein Stabilization. Pharmaceutical Development and Technology, 2007. 12(5): p. 505-523.
24. Alkhamis, K. A., Influence of Solid-State Acidity on the Decomposition of Sucrose in Amorphous Systems II (Effect of Buffer). Drug Development and Industrial Pharmacy, 2009. 35(4): p. 408-416.
25. Österberg, T. and T. Wadsten, Physical state of -histidine after freeze-drying and long-term storage. European Journal of Pharmaceutical Sciences, 1999. 8(4): p. 301-308.
26. Cun, D., et al., Preparation and characterization of poly(dl-lactide-co-glycolide) nanoparticles for siRNA delivery. International Journal of Pharmaceutics, 2010. 390(1): p. 70-75.
27. Katas, H., E. Cevher, and H. O. Alpar, Preparation of polyethyleneimine incorporated poly(d,l-lactide-co-glycolide) nanoparticles by spontaneous emulsion diffusion method for small interfering RNA delivery. International Journal of Pharmaceutics, 2009. 369(1-2): p. 144-154.
28. Zhang, J. and P. Ng, Composition, useful in a reconstituted composition for treating e.g. cancer, comprises a particle comprising many hydrophobic polymer-agent conjugates, and many hydrophilic-hydrophobic polymers, a surfactant, and a cyclic oligosaccharide, 2011, ZHANG J (ZHAN-Individual) NG P (NGPP-Individual). p. 265.
29. Miyata, K., et al., Freeze-dried formulations for in vivo gene delivery of PEGylated polyplex micelles with disulfide crosslinked cores to the liver. Journal of Controlled Release, 2005. 109(1-3): p. 15-23.
30. Troiano, G., et al., Lyophilized pharmaceutical composition for delivering therapeutic agent e.g. anticancer agent comprises polymeric nanoparticles, where upon reconstitution in aqueous medium the composition comprises microparticles of specific particle sizes, 2011, BIND BIOSCIENCES (BIND-Non-standard) TROIANO G (TROI-Individual) SONG Y (SONG-Individual) ZALE S E (ZALE-Individual) WRIGHT J (WRIG-Individual) VAN GEEN H T (VGEE-Individual). p. 63.
31. Zillies, J. C., et al., Formulation development of freeze-dried oligonucleotide-loaded gelatin nanoparticles. European Journal of Pharmaceutics and Biopharmaceutics, 2008. 70(2): p. 514-521.
32. Molina, M.d. C., S. D. Allison, and T. J. Anchordoquy, Maintenance of nonviral vector particle size during the freezing step of the lyophilization process is insufficient for preservation of activity: Insight from other structural indicators. Journal of Pharmaceutical Sciences, 2001. 90(10): p. 1445-1455.
33. Pfeifer, C., et al., Dry powder aerosols of polyethylenimine (PEI)-based gene vectors mediate efficient gene delivery to the lung. Journal of Controlled Release, 2011. 154(1): p. 69-76.
34. Brus, C., et al., Stabilization of oligonucleotide-polyethylenimine complexes by freeze-drying: physicochemical and biological characterization. Journal of Controlled Release, 2004. 95(1): p. 119-131.
35. Anwer, K., et al., New composition comprises a mixture of a cationic lipopolymer and a nucleic acid suspended in an aqueous solution, and a filler excipient, useful for gene delivery systems for transfecting a mammalian cell, 2009, EXPRESSION GENETICS INC (EXPR-Non-standard) MATAR M (MATA-Individual) FEWELL J (FEWE-Individual) LEWIS D H (LEWI-Individual) ANWER K (ANWE-Individual) EGEN INC (EGEN-Non-standard). p. 2178509-A2.
36. Kasper, J. C., et al., Development of a lyophilized plasmid/LPEI polyplex formulation with long-term stability—A step closer from promising technology to application. Journal of Controlled Release, 2011. 151(3): p. 246-255.
37. Csoergao, S. B. Z., et al., Pharmaceutical composition useful for treatment of e.g. allergic rhinitis, comprises nanoparticles composed of macromolecules e.g. antigen, and a linear polyethylenimine, in a liquid or lyophilized formulation, 2010, GENETIC IMMUNITY KFT (GENE-Non-standard). p. 65.
38. Lavertu, M., et al., High efficiency gene transfer using chitosan/DNA nanoparticles with specific combinations of molecular weight and degree of deacetylation. Biomaterials, 2006. 27(27): p. 4815-4824.
39. Methot, S., et al., Efficient in vivo gene delivery using chitosan/DNA nanoparticles for applications in cartilage repair. Osteoarthritis and Cartilage, 2007. 15, Supplement B(0): p. B74.
40. Jean, M., et al., Chitosan-plasmid nanoparticle formulations for IM and SC delivery of recombinant FGF-2 and PDGF-BB or generation of antibodies. Gene Therapy, 2009. 16(9): p. 1097-1110.
41. Ma, P. L., et al., New Insights into Chitosan-DNA Interactions Using Isothermal Titration Microcalorimetry. Biomacromolecules, 2009. 10(6): p. 1490-1499.
42. Thibault, M., et al., Intracellular Trafficking and Decondensation Kinetics of Chitosan-pDNA Polyplexes. Mol Ther, 2010.
43. Nimesh, S., et al., Enhanced Gene Delivery Mediated by Low Molecular Weight Chitosan/DNA Complexes: Effect of pH and Serum. Molecular Biotechnology, 2010: p. 1-15.
44. Thibault, M., et al., Excess polycation mediates efficient chitosan-based gene transfer by promoting lysosomal release of the polyplexes. Biomaterials, 2011. 32(20): p. 4639-4646.
45. Jean, M., et al., Chitosan-based therapeutic nanoparticles for combination gene therapy and gene silencing of in vitro cell lines relevant to type 2 diabetes. European Journal of Pharmaceutical Sciences, 2012. 45(1-2): p. 138-149.
46. Kim, D.-G., et al., Retinol-encapsulated low molecular water-soluble chitosan nanoparticles. International Journal of Pharmaceutics, 2006. 319(1-2): p. 130-138.
47. Sonaje, K., et al., Enteric-coated capsules filled with freeze-dried chitosan/poly(y-glutamic acid) nanoparticles for oral insulin delivery. Biomaterials, 2010. 31(12): p. 3384-3394.
48. Yin, L., et al., Drug permeability and mucoadhesion properties of thiolated trimethyl chitosan nanoparticles in oral insulin delivery. Biomaterials, 2009. 30(29): p. 5691-5700.
49. Motwani, S. K., et al., Chitosan-sodium alginate nanoparticles as submicroscopic reservoirs for ocular delivery: Formulation, optimisation and in vitro characterisation. European Journal of Pharmaceutics and Biopharmaceutics, 2008. 68(3): p. 513-525.
50. Seo, D.-H., et al., Methotrexate-incorporated polymeric nanoparticles of methoxy poly(ethylene glycol)-grafted chitosan. Colloids and Surfaces B: Biointerfaces, 2009. 69(2): p. 157-163.
51. Sung, H., et al., Composition, useful for e.g. lodging nanoparticles in target tissue to treat osteoporosis, comprises component of positively charged chitosan, component of negatively charged substrate and bioactive agent encapsulated within nanoparticles, 2011, GP MEDICAL INC (GPME-Non-standard) UNIV NAT TSING-HUA (UNTH). p. 39.
52. Mumper, R. J. and A. Rolland, Composition useful for delivery of nucleic acids or oligo:nucleotide(s) to cells—comprises the nucleic acid or oligonucleotide and a chitosan-based compound, 1997, GENEMEDICINE INC (GENE-Non-standard) VALENTIS INC (VALE-Non-standard) ROLLAND A (ROLL-Individual) MUMPER R J (MUMP-Individual). p. 914161-A2.
53. Tahara, K., et al., Improved cellular uptake of chitosan-modified PLGA nanospheres by A549 cells. International Journal of Pharmaceutics, 2009. 382(1-2): p. 198-204.
54. Okahata, S., et al., Molding DNA/chitosan complex, involves filling DNA/chitosan complex in mold, supplying buffer to complex, freeze drying the complex containing buffer, and obtaining DNA/chitosan complex molded according to shape of mold, 2007, OKAHATA Y (OKAH-Individual) FUKUSHIMA T (FUKU-Individual) NICHIRO KK (NCHR) OKAHATA S (OKAH-Individual). p. 10.

1. A polyelectrolyte complex composition comprising a polymer, a nucleic acid molecule, a lyoprotectant, and a buffer, said composition preserving biological activities of the polyelectrolyte complex following freeze-drying and rehydration.

2. The polyelectrolyte complex composition according to claim 1, said composition having a Z-average below about 750 nm following freeze-drying and rehydration.

3. The polyelectrolyte complex composition according to claim 1, said composition being substantially free of aggregation following freeze-drying and rehydration.

4. The polyelectrolyte complex composition according to claim 1, said composition having a polydispersity index which is at most 0.5 following freeze-drying and rehydration.

5. The polyelectrolyte complex composition according to claim 1, said polyelectrolyte complex achieving at least about 10% transfection level following freeze-drying and rehydration.

6. The polyelectrolyte complex composition according to claim 1, said composition being reconstituted within about 10 minutes following freeze-drying and rehydration.

7. (canceled)

8. The polyelectrolyte complex composition according to claim 1, said composition being near iso-osmolality following freeze-drying and rehydration.

9. (canceled)

10. The polyelectrolyte complex composition according to claim 1, said composition having a near neutral pH following freeze-drying and rehydration.

11. (canceled)

12. The polyelectrolyte complex composition according to claim 1, being freeze-dried.

13. The polyelectrolyte complex composition according to claim 1, wherein the polymer is chitosan.

14. The polyelectrolyte complex composition according to claim 13, wherein the chitosan number average molecular weight (Mₙ) is between 4 and 200 kDa.

15. The polyelectrolyte complex composition according to claim 13, wherein the chitosan Mₙ is between 10 and 80 kDa.

16. The polyelectrolyte complex composition according to claim 13, wherein the chitosan degree of deacetylation (DDA) is between 70 and 100%.

17. (canceled)

18. The polyelectrolyte complex composition according to claim 3, wherein chitosan/nucleic acid N/P ratio is between 1.2 and 30.

19.-20. (canceled)

21. The polyelectrolyte complex composition according to claim 1, wherein the nucleic acid molecule is at least one of a DNA, a plasmid (pDNA), a minicircle, an oligodeoxynucleotide (ODN), and a ribonucleic acid molecule.

22. (canceled)

23. The polyelectrolyte complex composition according to claim 1, wherein the lyoprotectant is a disaccharide, a trisaccharide, a oligosaccharide/polysaccharide, a polyol, a polymer, a high molecular weight excipient, an amino acid molecule or any combination thereof.

24. The polyelectrolyte complex composition according to claim 23, wherein the disaccharide is at least one of sucrose, trehalose, lactose, maltose, cellobiose, and melibiose.

25. The polyelectrolyte complex composition according to claim 23, wherein disaccharide concentration is between 0.1 and 10% (w/v).

26.-27. (canceled)

28. The polyelectrolyte complex composition according to claim 23, wherein the trisaccharide is at least one of maltotriose and raffinose.

29. The polyelectrolyte complex composition according to claim 23, wherein trisaccharide concentration is between 0.1 and 10% (w/v).

30.-31. (canceled)

32. The polyelectrolyte complex composition according to claim 23, wherein the oligosaccharide/polysaccharide is at least one of dextran, cyclodextrin, maltodextrin, hydroxyethyl starch, ficoll, cellulose, hydroxypropylmethyl cellulose, and inulin.

33.-34. (canceled)

35. The polyelectrolyte complex composition according to claim 23, wherein oligosaccharide/polysaccharide concentration is between 0.1 and 10% (w/v).

36.-38. (canceled)

39. The polyelectrolyte complex composition according to claim 23, wherein the polyol concentration is between 0.1 and 10% (w/v).

40.-41. (canceled)

42. The polyelectrolyte complex composition according to claim 23, wherein the amino acid molecule is at least one of lysine, arginine, glycine, alanine and phenylalanine.

43. The polyelectrolyte complex composition according to claim 23, wherein the amino acid molecule concentration is between 1 and 100 mM.

44.-46. (canceled)

47. The polyelectrolyte complex composition according to claim 23, wherein the high molecular weight excipient is at least one of PEG, gelatin, polydextrose and PVP.

48. The polyelectrolyte complex composition according to claim 1, wherein the buffer is at least one of sodium citrate, histidine, sodium malate, sodium tartrate and sodium bicarbonate.

49.-57. (canceled)

58. A polyelectrolyte complex composition comprising a chitosan, a deoxyribonucleic acid in an amount of about 50 μg/mL, trehalose in an amount of between about 0.5% (w/v) and about 1% (w/v) and histidine in an amount of between about 3 mM and about 4 mM.

59.-75. (canceled)
